(19) United States
(12) Patent Application Publication
(10) Pub. No.: US 2024/0268394 A1
(43) Pub. Date: Aug. 15, 2024

(54) BACTERIAL STRAINS THAT ENHANCE CROP LEGUME PLANT GROWTH

(71) Applicant: VIRGINIA TECH INTELLECTUAL PROPERTIES, INC., Blacksburg, VA (US)

(72) Inventors: Mark A. Williams, Blacksburg, VA (US); Roland Griggs, Christiansburg, VA (US); Hazem Sharaf, Bkacksburg, VA (US); Connor Doyle, Blacksburg, VA (US)

(21) Appl. No.: 18/567,887

(22) PCT Filed: Jun. 8, 2022
(86) PCT No.: PCT/US2022/072811

§ 371 Date: Dec. 7, 2023

Related U.S. Application Data
(60) Provisional application No. 63/208,208, filed on Jun. 8, 2021.

(51) Int. Cl. A01N 63/27; A01P 21/00; C12N 1/20; C12R 1/38

(52) U.S. Cl. CPC A01N 63/27; A01P 21/00; C12N 1/205; C12R 2001/38

(57) ABSTRACT

Disclosed herein are compositions containing Pseudomonas strains referred to herein as Bullseye and Pancake and/or extracts from Pseudomonas strain cultures and methods of making and using the same. Also disclosed herein are methods for increasing crop legume plant growth using the disclosed compositions. The compositions are effective as well as environmentally benign and are not detrimental to human or animal health. Also disclosed are methods for extracting plant growth promoting rhizobacteria (PGPR) from nodules of a soybean plant.

### CROSS-REFERENCE TO RELATED APPLICATIONS

This application claims benefit of U.S. Provisional Application No. 63/208,208, filed Jun. 8, 2021, which is hereby incorporated herein by reference in its entirety.

### SEQUENCE LISTING

This application contains a sequence listing filed in electronic form as an ASCII.txt file entitled “222204_2580_Sequence_Listing_ST25” created on Jun. 8, 2022 and having 33,359,087 bytes. The content of the sequence listing is incorporated herein in its entirety.

### BACKGROUND

Soybeans are one of the most commonly grown crops in the world, and nitrogen-fixing bacteria colonize the roots of soybeans and initiate the formation of spherical nodules attached to the roots. Inside the nodules, these bacteria convert atmospheric nitrogen to plant-available forms in exchange for sugar from the plant, and such bacteria reduce the need to add nitrogen fertilizer to agricultural fields. Other non-nitrogen-fixing bacteria also reside in nodules, but their role in the nodule is not well understood.

### SUMMARY

In accordance with the purpose(s) of the present disclosure, as embodied and broadly described herein, the disclosure, in one aspect, relates to compositions containing Pseudomonas strains referred to herein as Pseudomonas nodulensis MW1 (Pancake, PAMW1), Pseudomonas nodulensis MW2 (Bullseye, BUMW2), Pseudomonas nodulensis MW3 (Starfish, STMW3), Pseudomonas nodulensis MW4 (Jellyfish, JEMW4), respectively.

In some embodiments, the Pseudomonas nodulensis MW1 (Pancake, PAMW1) microbial strain is the strain deposited with the WDCM as WDCM1247. In some embodiments, the Pseudomonas nodulensis MW2 (Bullseye, BUMW2) microbial strain is the strain deposited with the WDCM as WDCM1248. In some embodiments, the Pseudomonas nodulensis MW4 (Jellyfish, JEMW4) microbial strain is the strain deposited with the WDCM as WDCM1247.

Therefore, also disclosed herein is a composition comprising a microbial strain comprising a genome having DNA sequences exhibiting at least 85, 86, 87, 88, 89, 90, 91, 92, 93, 94, 95, 96, 97, 98, 99, 99.5, or 100% sequence identity to one or more of SEQ ID NOs: 1-31 (Pancake, PAMW1) or an extract produced by culturing the microbial strain, or any combination thereof; and at least one excipient, diluent, or carrier.

Also disclosed herein is a composition comprising a microbial strain comprising a genome having DNA sequences exhibiting at least 85, 86, 87, 88, 89, 90, 91, 92, 93, 94, 95, 96, 97, 98, 99, 99.5, or 100% sequence identity to one or more of SEQ ID NO:32-76 (Bullseye, BUMW2), or an extract produced by culturing the microbial strain, or any combination thereof; and at least one excipient, diluent, or carrier.

Also disclosed herein is a composition comprising a microbial strain comprising a genome having DNA sequences exhibiting at least 85, 86, 87, 88, 89, 90, 91, 92, 93, 94, 95, 96, 97, 98, 99, 99.5, or 100% sequence identity to one or more of SEQ ID NO:77-107 (Starfish, STMW3), or an extract produced by culturing the microbial strain, or any combination thereof; and at least one excipient, diluent, or carrier.

Also disclosed herein is a composition comprising a microbial strain comprising a genome having DNA sequences exhibiting at least 85, 86, 87, 88, 89, 90, 91, 92, 93, 94, 95, 96, 97, 98, 99, 99.5, or 100% sequence identity to one or more of SEQ ID NO: 108-137 (Jellyfish, JEMW4), or an extract produced by culturing the microbial strain, or any combination thereof; and at least one excipient, diluent, or carrier.

Also disclosed are crop legume seeds, such as soybean or edamame seeds, coated with the disclosed compositions.

Also disclosed herein are methods for increasing crop legume plant growth using the disclosed compositions. The compositions are effective as well as environmentally benign and are not detrimental to human or animal health.

Also disclosed herein are methods for extracting plant growth promoting rhizobacteria (PGPR) from nodules of a crop legume plant.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. Colony morphologies of bacteria used in this study: (A) Bullseye, (B) Pancake, and (C) Starfish grown on, modified LB agar (blue mark is permanent marker on the outside of the plate, not part of the colony).

FIG. 2. Experimental design showing the treatment types and number of treatment repetitions for each soybean cultivar.

FIG. 3. Principal coordinate analysis of Bray-Curtis dissimilarity from the 1st harvest showing the effect of, treatments on indices growth, yield, and nitrogen fixation. Figures are labeled as follows: (A) unrelativized Asgrow, data, (B) relativized Asgrow data, (C) unrelativized Pioneer data, and (D) relativized Pioneer data (D) Treatments, are labeled according to cultivar (Ag=Asgrow, Pi=Pioneer) and Bacteria (B=Bullseye, P=Pancake, S=, Starfish, C=Control).

FIG. 4. Canonical correspondence analysis of unrelativized data from 1st harvest showing the effect of treatments, on indices of growth, yield, and nitrogen fixation. Figures are labeled as follows: (A) Asgrow (B) Pioneer, Treatments are labeled according to cultivar (Ag=Asgrow, Pi=Pioneer) and Bacteria (B=Bullseye, P=, Pancake, S=Starfish, C=Control).

FIG. 5. Mean values of first harvest response variables that showed significant differences between cultivars: (A), SPAD, (B) stomatal conductance, and (C) nodule number per plant for Asgrow (Ag) and Pioneer (Pi) soybeans, following 1st harvest.

FIG. 6. Mean root length of soybeans treated with Bullseye (B), Pancake (P), Starfish, (S) and uninoculated, controls (C). Bars indicate standard error.

FIG. 7. Bray-Curtis ordination plots for the 2nd Harvest showed the effect of treatments on indices of growth, yield, and nitrogen fixation. Figures are labeled as follows: (A) unrelativized Asgrow data, (B) relativized Asgrow, data, (C) unrelativized Pioneer data, and (D) relativized Pioneer data (D) Treatments are labeled according to, cultivar (Ag=Asgrow, Pi=Pioneer) and Bacteria (B=Bullseye, P=Pancake, S=Starfish, C=Control).

FIG. 8. Canonical correspondence analysis of unrelativized data from 2nd harvest showing the effect of treatments on indices of growth, yield, and nitrogen fixation. Figures are labeled as follows: (A) Asgrow (B) Pioneer. Treatments are labeled according to cultivar (Ag=Asgrow, Pi=Pioneer) and Bacteria (B=Bullseye, P=Pancake, S=Starfish, C=Control).

FIG. 9. Mean values of second harvest response variables that showed significant differences between cultivars: (A) SPAD, (B) height, (C) root wet mass, (D) nodules per plant, (E) root dry mass, (F) shoot-to-root mass ratio, (G) seeds per plant.

FIG. 10. (A) A representative blue CAS agar plate two days after inoculation showing all three inoculants solubilize Fe as indicated by the orange halos surrounding the colonies. Starting in the top right quadrant and moving in a clockwise direction, inoculant strains are positioned as follows: Control, Bullseye, Pancake, Starfish. (B) Transparent halos surrounding colonies grown on a representative NBRIP plate 14 days after inoculation indicate all three strains solubilize P. The relative order of bacteria on the plate is the same as that in image A.

FIG. 11. Comparison of average treatment means for plants treated with Bullseye (B), Pancake (P), Starfish (S) and controls (C). Treatment means for each response variable measured during the 1st harvest are shown: (A) SPAD, (B) stomatal conductance, (C) height, (D) flowers per plant, (E) trifoliates per plant, (F) trifoliate length, (G) volumetric water content, (H) root length, (I) root wet mass, (J) shoot wet mass, (K) nodules per plant, (L) nodule mass, (M) total wet mass, (N) shoot dry mass, (O) root dry mass, (P) total dry mass, and (Q) shoot-to-root mass ratio.

FIG. 12. Second Harvest comparison of average treatment means for plants treated with Bullseye (B), Pancake (P), Starfish (S) and controls (C). Treatment means for each response variable measured during the 2nd harvest are shown: (A) SPAD, (B) height, (C) pods per plant, (D) root length, (E) root wet mass, (F) shoot wet mass, (G) nodules per plant, (H) nodule mass, (I) total wet mass, (J) shoot dry mass, (K) root dry mass, (L) total dry mass, (M) shoot-to-root mass ratio, (N) pod wet mass, (O) seeds per plant, (P) grain dry mass, (Q) number of auxiliary branches, (R) aboveground wet mass, (S) pod dry mass, (T) aboveground dry mass, (U) seed oil content, and (V) seed protein content.

### DETAILED DESCRIPTION

Disclosed herein are compositions and methods for promoting soybean plant growth, the methods including applying to a soybean seed or plant an effective amount of a composition, wherein the composition includes a microbial strain comprising a DNA sequence exhibiting 85, 86, 87, 88, 89, 90, 91, 92, 93, 94, 95, 96, 97, 98, 99, 99.5, or 100% whole genome average nucleotide sequence identity or more to at least one of SEQ ID NOs: 1-2, an extract produced by culturing the microbial strain, or any combination thereof. In a further aspect, the microbial strain can be related to Pseudomonas azotoformans strain Pancake and/or Pseudomonas azotoformans strain Bullseye derived from soybean cultivar 5002T. These strains are referred to herein as Pseudomonas nodulensis MW1 and Pseudomonas nodulensis MW2, respectively. In some embodiments, the Pseudomonas nodulensis MW1 microbial strain is the strain deposited with the WFCC as WDCM1247. In some embodiments, the Pseudomonas nodulensis MW2 microbial strain is the strain deposited with the WFCC as WDCM1248.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

### Definitions

Prior to describing the various aspects of the present disclosure, the following definitions are provided and should be used unless otherwise indicated. Additional terms may be defined elsewhere in the present disclosure.

It must be noted that, as used in the specification and the appended claims, the singular forms “a,” “an,” and “the” include plural referents unless the context clearly dictates otherwise.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a plant,” “a leaf,” or “an excipient,” includes, but is not limited to, collections, mixtures, or combinations of two or more such plants, leaves, or excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of an active ingredient refers to an amount that is sufficient to achieve the desired improvement in the property modulated by the formulation component, e.g. achieving the desired level of growth promotion. The specific level in terms of wt % in a composition required as an effective amount will depend upon a variety of factors including the amount and type of the herein described Pseudomonas species metabolites in the composition and/or presence of live cells, amount and type of any carriers or excipients, conditions surrounding the plants to be treated, length of time since treatment and/or number of treatments to be applied, degree of infestation, and identity of the pathogen to be treated.

As used herein, the terms “optional” or “optionally” means that the subsequently described event or circumstance can or cannot occur, and that the description includes instances where said event or circumstance occurs and instances where it does not.

“Inoculum” as used herein refers to a composition containing microorganisms, wherein the composition used to pretreat a plant for the purpose of establishing a population of the microorganisms on the plant. A successful inoculum can be at an active growth stage and size and will generally be free from contamination and may include additional components useful for establishing a population of microorganisms such as, for example, culture medium, solvents, buffers, and the like. In some aspects, a bacterial inoculum is prepared to infect plants in order to assess the efficacy of disclosed treatments. In other aspects, other forms of inoculum can be used to treat plants to increase plant growth under typical or conditions of the environment that cause plant stress.

“Colony forming units” (CFU) refers to an estimate of the number of viable microorganisms (e.g., bacteria) in a sample. In one aspect, number of CFU in a sample can be established by culturing the sample on a plate and counting microbial colonies, wherein each colony is assumed to have arisen from a single cell or group of cells.

As used herein, the term “prevent” or “preventing” refers to precluding, averting, obviating, forestalling, stopping, or hindering a plant disease from infecting a plant or spreading among a plant population, especially by advance action. It is understood that where reduce, inhibit or prevent are used herein, unless specifically indicated otherwise, the use of the other two words is also expressly disclosed. In one aspect, prevention of the plant disease is associated with reduced transmission of the plant disease, either by (i) stopping the spread of the disease from one part of a plant to the rest of the plant or (ii) stopping the spread of the disease from one plant to a nearby plant. Reduced transmission and prevention can be assessed quantitatively based on knowledge in the art such as plant growth habit, conditions for disease spread in a given installation type, and the like, wherein reduced transmission or prevention results in a lower amount of spread of a disease than would ordinarily be expected.

As used herein, “increase” or “increasing” refers to making something greater in size, amount, length, or the like. Thus, in one aspect, a treatment that increases plant growth leads to an improvement in at least one growth-related quality of the plant compared to an untreated plant (e.g., the plant with increased growth is larger in size, has greater foliage area, produces more fruits, or has a longer lifetime than an untreated counterpart).

Unless otherwise specified, temperatures referred to herein are based on atmospheric pressure (i.e. one atmosphere).

### Microbial Compositions and Extracts

In one aspect, disclosed herein are compositions including a Pseudomonas Pseudomonas azotoformans strain Bullseye and/or Pancake derived from soybean cultivar 5002T that has a DNA sequence exhibiting about 85, 86, 87, 88, 89, 90, 91, 92, 93, 94, 95, 96, 97, 98, 99, 99.5, or 100% average nucleotide sequence identity to at least one of SEQ ID NOs: 1-2, or any range encompassing any of the foregoing values. In one aspect, SEQ ID NOs: 1-2 represent contigs of the genome sequence of SSG. Further in this aspect, SEQ ID NOs: 1-2 can be assembled in a sequential order to identify or elucidate one or more portions of the genome of SSG.

In another aspect, the microbial strain can be frozen, lyophilized, or present as metabolically active cells. In one aspect, the microbial strain is present in the composition in an amount of from about 109 to about 103 colony forming units (CFU) per mL, or at about 109, 108, 107, 106, 105, 104, or about 103 CFU/mL, or a combination of any of the foregoing values, or a range encompassing any of the foregoing values.

In yet another aspect, disclosed herein are extracts produced by culturing the disclosed microbial strains and/or microbial compositions. In one aspect, the extracts are substantially free of microbial cells. In another aspect, the extracts can be produced by (a) culturing the microbial strain in a culture medium and (b) filtering the culture medium. In one aspect, the culture medium can be filtered with a 0.22 μm filter

### Excipients, Diluents, Carriers, and Additional Active Ingredients

In any of these aspects, the compositions further include at least one excipient, diluent, or carrier, or any combination thereof. In another aspect, at least one excipient, diluent, or carrier can be a surfactant, a solvent, an emulsifier, a buffer, a cryoprotectant, a salt, microbial culture medium, a wetting agent, a bulking agent, an anti-caking agent, a thickener, a clay, a mineral, a lipid, a gum, a dye or colorant, a biological waste material, or any combination thereof. In some aspects, one compound or component can fit in different categories (e.g., a clay can also act as a thickener and/or a bulking agent, or a salt may also have buffering properties and/or act as a cryoprotectant, and the like).

In one aspect, the cryoprotectant can be ethylene glycol, propylene glycol, glycerol, dimethyl sulfoxide, sucrose, trehalose, or any combination thereof.

In one aspect, the clay can be a natural clay, a clay mineral, or a natural or synthetic silicate salt. In a further aspect, the clay can be selected from aluminum magnesium silicate, aluminum potassium sodium silicate, aluminum silicate, aluminum sodium silicate, attapulgite-type clay, bentonite, calcium oxide silicate, calcium silicate, Fuller's earth, kaolin, magnesium oxide silicate, magnesium silicate, magnesium silicate hydrate, montmorillonite, perlite, potassium aluminum silicate, vermiculite, wollastonite, zeolites, or any combination thereof.

In another aspect, the salt can be a salt, buffer, or any combination thereof. In one aspect, the salt can be calcium acetate, calcium citrate, calcium sulfate, citric acid, dipotassium citrate, disodium citrate, disodium sulfate, ferric oxide, ferrous oxide, iron magnesium oxide, magnesium carbonate, magnesium oxide, magnesium sulfate, potassium acetate, potassium bicarbonate, potassium chloride, potassium citrate, potassium sulfate, sodium acetate, sodium bicarbonate, sodium chloride, sodium citrate, sodium sulfate, zinc iron oxide, zinc oxide, zinc stearate, hydrates thereof, conjugate acids and/or bases thereof, and any combination thereof.

In another aspect, the mineral can be calcium carbonate, feldspar, granite, graphite, gypsum, hematite, lime, limestone, mica, mica-group minerals, nepheline syenite, pumice, shale, or any combination thereof.

In one aspect, the lipid can be one or more waxes, one or more acylglycerols, one or more triglycerides, one or more diglycerides, one or more monoglycerides, one or more fatty acids, one or more steroids, or any combination thereof. Examples of useful lipids include, but are not limited to, tristearin, glycerol behenate, glycerol monostearate, stearic acid, cholesterol, cetyl palmitate, and combinations thereof. In another aspect, the lipid can further function as an emulsifier, surfactant, detergent, wetting agent, foaming agent, dispersant, or any combination thereof.

In one aspect, the dye or colorant can be selected from chlorophyll, red cabbage color, ultramarine blue, or any combination thereof.

In another aspect, the thickener can be agar, carrageenan, or any combination thereof. In another aspect, the wax can be beeswax, carnauba wax, paraffin wax, or any combination thereof. In still another aspect, the gum can be locust bean gum, gellan gum, guar gum, gum arabic, gum tragacanth, xanthan gum, or any combination thereof.

In one aspect, the biological waste material can be almond hulls, almond shells, bone meal, bran, bread crumbs, cardboard, cellulose or a chemically-modified cellulose, citrus meal, citrus pulp, clam shells, cocoa, cocoa shell flour, cocoa shells, coffee grounds, cork, corn cobs, cracked wheat, diatomaceous earth, Douglas fir bark, egg shells, fish meal, peanut shells, peat moss, red cedar chips, sawdust, soybean hulls, soybean meal, soybean flour, walnut flour, walnut shells, wheat, or any combination thereof.

In some aspects, the surfactant can be a polysorbate such as, for example, polysorbate 20. In another aspect, the solvent can be water. In one aspect, the at least one excipient, diluent, or carrier can confer increased stability, wettability, dispersibility, or adherence to a substrate relative to a composition lacking the carrier.

In one aspect, the composition can be or include an emulsion, colloid, granule, pellet, powder, spray, suspension, or solution.

In another aspect, the composition can further include at least one additional active ingredient. In a further aspect, the additional active ingredient can be a fertilizer, a pesticide, an herbicide, or any combination thereof.

### Method for Increasing Plant Growth

Disclosed herein is a method for increasing plant growth of a crop leguminous crop, such as a soybean or edamame, the method including applying the disclosed compositions to a plant so that the treated plant has increased growth compared to an untreated plant. In some embodiments, the composition can be applied to the roots, leaves, fruits, flowers, stems, or seeds of the plant, or any combination thereof. In still another aspect, the composition can be applied to soil, compost, mulch, leaf litter, sawdust, straw, pine straw, wood chips, gravel, plant growing medium, or other material in a bed surrounding the plant.

In some embodiments, increasing plant growth can result in increased biomass of the treated plant compared to an untreated plant. In another aspect, increasing plant growth can result in increased fruit production of the treated plant compared to an untreated plant. In still another aspect, increasing plant growth can lead to increased production period of the treated plant compared to an untreated plant, or to an increased productive lifespan of the treated plant compared to an untreated plant. In yet another aspect, increasing plant growth can result in an increased foliage area of the treated plant compared to an untreated plant, wherein increased foliage area can include a greater number of leaves, a larger surface area per individual leaf, or any combination thereof. In one aspect, plant growth can be increased by from at least 35% to at least 75% compared to an untreated plant, or by about 35, 40, 45, 50, 55, 60, 65, 70, or about 75% compared to an untreated plant, or a combination of any of the foregoing values, or a range encompassing any of the foregoing values. In one aspect, plant growth increase by about 35%, about 55%, or about 75% compared to an untreated plant.

In still another aspect, disclosed herein is a method for increasing plant growth, the method including the steps of applying to a plant or to a plant growing medium an effective amount of a composition, wherein the composition includes a microbial strain that includes a DNA sequence exhibiting at least 85% sequence identity to at least one of SEQ ID NOs: 1-2, an extract produced by culturing the microbial strain, or any combination thereof, so that a treated plant has increased growth compared to an untreated plant. In another aspect, the DNA sequence can have from about 95% to about 100% sequence identity to at least one of SEQ ID NOs: 1-2, or about 95, 96, 97, 98, 99, 99.5, or about 100% sequence identity, or a combination of any of the foregoing values, or a range encompassing any of the foregoing values. Also disclosed are plants treated by the disclosed methods.

Method for Obtaining Growth Promoting Pseudomonas Derived from Soybean Cultivar 5002T

Also disclosed herein is a method for extracting plant growth promoting rhizobacteria (PGPR) from nodules of a soybean plant. In some embodiments, the soybean plant is a 5002T cultivar, or derivative thereof.

The method involves the extraction of nodule endophytes for cultivation and isolation. In some embodiments, the method first involves mechanically cleaning the external surface of the nodules to remove soil and debris without damaging the nodule barrier that protects the nodule interior. The method can then involve sterilizing the surface of the nodules with a bleach and ethanol solution to lyse and kill bacterial cells and denature DNA. The method can then involve washing the nodules to remove the bleach and ethanol by repeated washings. The method can then involve testing surface of nodules to ensure sterility, wherein if the nodules are not shown to be sterile then repeat sterilization and washing steps. The method can then involve extracting endophytes from the nodule and plating them on KBC to grow Pseudomonas spp. Note that other variations of KB media did not provide for the selective growth and diversity of colony types, so KBC is the media of choice for growth on plates and in solution. Finally, the method can involve selecting colonies for Pseudomonas with different morphologies and streaking these colonies to isolate PGPR bacterial variants.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered Aspects, which should not be confused with the claims.

Aspect 1. A composition comprising a microbial strain comprising a DNA sequence exhibiting at least 85% sequence identity to at least one of SEQ ID NOs: 1-2, an extract produced by culturing the microbial strain, or any combination thereof; and at least one excipient, diluent, or carrier.

Aspect 2. The composition of aspect 1, wherein the microbial strain is deposited with the WFCC as WDCM1247 or WDCM1248.

Aspect 3. The composition of aspect 1, wherein the DNA sequence exhibits at least 95% sequence identity to at least one of SEQ ID NO: 1 or 2.

Aspect 4. The composition of aspect 1, wherein the DNA sequence exhibits at least 99% sequence identity to at least one of SEQ ID NO:1 or 2.

Aspect 5. The composition of aspect 1, wherein the DNA sequence exhibits 100% sequence identity to at least one of SEQ ID NO: 1 or 2.

Aspect 6. The composition of any one of aspects 1 to 5, wherein the microbial strain is frozen, lyophilized, or is present as metabolically active cells.

Aspect 7. The composition of any one of aspects 1 to 6, wherein the microbial strain is present in an amount of from about 109 to about 103 colony forming units per mL.

Aspect 8. The composition of any one of aspects 1 to 6, wherein the microbial strain is present in an amount of from about 109 to about 108 colony forming units per mL.

Aspect 9. The composition of any of aspects 1 to 8, wherein the at least one excipient, diluent, or carrier confers increased stability, wettability, dispersibility, or adherence to a substrate relative to a composition lacking the carrier.

Aspect 10. The extract of any one of aspects 1 to 9, wherein the extract is produced by:
(a) culturing the microbial strain in a culture medium; and
(b) filtering the culture medium.

Aspect 11. The composition of any one of aspects 1 to 10, wherein the composition comprises an emulsion, a colloid, a granule, a pellet, a powder, a spray, a suspension, or a solution.

Aspect 12. The composition of any one of aspects 1 to 11, wherein the at least one excipient, diluent, or carrier comprises a surfactant, a solvent, an emulsifier, a buffer, a cryoprotectant, a salt, microbial culture medium, a wetting agent, a bulking agent, an anti-caking agent, a thickener, a clay, a mineral, a lipid, a gum, a dye or colorant, a biological waste material, or any combination thereof.

Aspect 13. The composition of aspect 12, wherein the surfactant comprises polysorbate 20.

Aspect 14. The composition of aspect 12 or 13, wherein the solvent comprises water.

Aspect 15. The composition of any one of aspects 1 to 14, wherein the composition further comprises an effective amount of at least one additional active ingredient.

Aspect 16. The composition of aspect 15, wherein the additional active ingredient comprises a fertilizer, a pesticide, an herbicide, or any combination thereof.

Aspect 17. A crop legume seed coated with the composition of any one of aspects 1 to 16.

Aspect 18. The crop legume seed of aspect 17, wherein the seed is a soybean or edamame seed.

Aspect 19. The crop legume seed of aspect 17 or 18, wherein the composition further comprises a binder, filler, adhesives, adjuvant, thickener, or any combination thereof.

Aspect 20. A method for increasing crop legume plant growth, the method comprising applying the composition of any one of aspects 1 to 16 to a soybean plant or seed, so that the treated plant or plant derived from the treated seed has increased growth compared to an untreated plant or plant derived from an untreated seed.

Aspect 21. The method of aspect 20, wherein the crop legume plant is a soybean or edamame plant.

Aspect 22. The method of aspect 21, wherein the soybean plant is a Glycine max species.

Aspect 23. The method of any one of aspects 20 or 22, wherein the composition is applied to the roots, leaves, fruits, flowers, stems, or seeds of the plant, or any combination thereof.

Aspect 24. The method of any one of aspects 20 to 23, wherein the composition is applied to soil, compost, mulch, leaf litter, sawdust, straw, pine straw, wood chips, gravel, plant growing medium, or other material in a bed surrounding the plant.

Aspect 25. The method of any one of aspects 20 to 24, wherein increasing soybean plant growth comprises increased biomass of the treated plant compared to an untreated plant.

Aspect 26. The method of any one of aspects 20 to 25, wherein increasing soybean plant growth comprises increased bean production of the treated soybean plant compared to an untreated plant.

Aspect 27. The method of any one of aspects 20 to 26, wherein increasing soybean plant growth comprises increased production period of the treated soybean plant compared to an untreated soybean plant.

Aspect 28. The method of any one of aspects 20 to 27, wherein performing the method increases soybean plant growth by at least 10% for the treated soybean plant or the plant derived from the treated seed compared to an untreated soybean plant or plant derived from an untreated seed.

Aspect 29. The method of any one of aspects 20 to 27, wherein performing the method increases soybean plant growth by at least 55% for the treated soybean plant or the plant derived from the treated seed compared to an untreated soybean plant or plant derived from an untreated seed.

Aspect 30. The method of any one of aspects 20 to 27, wherein performing the method increases soybean plant growth by at least 75% for the treated soybean plant or the plant derived from the treated seed compared to an untreated soybean plant or plant derived from an untreated seed.

Aspect 31. A plant treated using the method of any one of aspects claims 20 to 30.

Aspect 32. A method for extracting plant growth promoting rhizobacteria (PGPR) from nodules of a crop legume plant, comprising
(a) mechanically cleaning the external surface of the nodules to remove soil and debris without damaging the nodule barrier that protects the nodule interior;
(b) sterilizing the surface of the nodules with a bleach and ethanol solution to lyse and kill bacterial cells and denature DNA;
(c) washing the nodules to remove the bleach and ethanol by repeated washings;
(d) testing surface of nodules to ensure sterility, wherein if the nodules are not shown to be sterile then repeat sterilization and washing steps;
(e) extracting endophytes from the nodule and plating them on KBC to grow Pseudomonas spp.; and
(f) selecting colonies for Pseudomonas with different morphologies and streaking these colonies to isolate PGPR bacterial variants.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Pseudomonas Spp. Isolated from Soybean Nodules Promote Soybean Growth and Nitrogen Fixation

### Materials and Methods

### Bacteria Isolation and Identification

Bacteria were isolated from nodules of the soybean cultivar 5002T after sterilizing the outside of nodules with sodium hypochlorite. Detailed methods for sterilization are outlined in Sharaf et al. (2019). After sterilization, the outside of select nodules were streaked onto Congo Red media to test whether or not bacteria survived the sterilization process. Sterilized nodules and 2 mL of 0.9 M NaCl solution were added to 5 ml conical tubes. A sterile rod was used to crush the nodules in the tube to suspend the bacteria from inside the nodules in solution. The tubes were then vortexed and given approximately 5 minutes to allow the nodule particles to settle prior to being stored at 4° C.

Bacteria suspended in the solution were then streaked onto plates containing KBC media (Mohan and Schaad 1987), a modified version of King's B media (King et al. 1954), to select for Pseudomonas spp. Colonies displaying different morphologies were selected and re-streaked onto new plates until new plates contained only one colony morphology. A total of four colony morphologies were identified. For the sake of clarity, bacteria displaying these four colony morphologies were named according to distinguishing features and will be referred to in this paper as Bullseye, Pancake, Starfish, and Jellyfish respectively. Jellyfish was not used during this study.

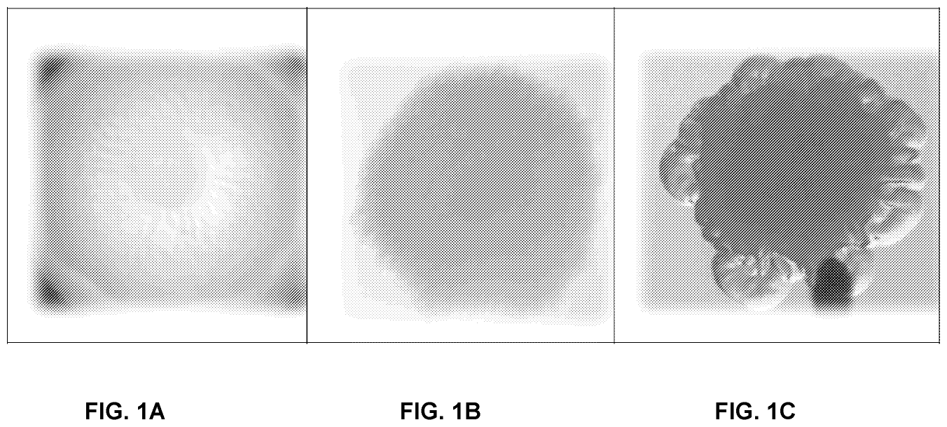

To confirm all isolated strains were in fact Pseudomonas spp., the 16 s rRNA and citrate synthase (CTS) genes were amplified then sequenced by Virginia Tech's Biocomplexity Institute (Blacksburg, VA, USA). Genomic analysis revealed all four strains were closely related members of the genus Pseudomonas spp., but it did not allow us to determine the exact species or strains of each bacterium. Consequently, differences in colony morphology were deemed sufficient evidence for treating each bacterium as a different strain during the experiment until full genome sequencing could be conducted. Pictures of each of the strains used for inoculation is shown in FIG. 1.

### Soil Preparation, Potting, Planting, and Inoculation

Field soil from Virginia Tech's Kentland Farm (37.1983ºN, 80.5747° W) was collected to be used as potting media during the experiment. The soil was taken from a field in which soybeans were grown the previous year thus ensuring an established population of Bradyrhizobium spp. was present. Additionally, soil was collected from the top 20 cm of a silt loam soil with a pH of approximately 6.0 (1:1 water/soil v/w). The soil was then mixed with perlite in approximately a 2:3 ratio by volume, and the soil-perlite mix was then used to fill 80, 5.68 L (trade size #2) pots. Approximately 800 mL of 1:1 sand:vermiculite mix was then spread on the top of the soil-perlite mix to reduce weed growth and moisture evaporation.

Two commercially available varieties of soybean seed, Asgrow AG46X6 and Pioneer P48A60X, were germinated by wrapping 6 seeds in moist paper towels and placing them in open plastic bags for 4 days. These cultivars were selected, because they are commonly grown agronomic varieties in Virginia. Three germinated seeds were placed an equal distance apart between the soil-perlite layer and sand-vermiculite layer of each pot, and forty pots contained Asgrow AG46X6 while the other forty pots contained Pioneer P48A60X. Plants were grown in a greenhouse with temperatures set at 28° C. during the day and 21° C. at night. An automatic irrigation system was also used to water the plants. Of note, cultivars P48A60X and AG46X6 fall into maturity groups 4.8 and 4.6 respectively, and planting occurred on July 13th rather than a more ideal time in the spring due to logistical challenges.

Three of the four bacteria isolated from soybean nodules, Bullseye, Pancake, and Starfish were selected to serve as inoculants. Each strain was grown overnight in 500 mL of modified lysogeny broth (LB) (Bertani 1951) (10 g NaCl and 5 g yeast extract per liter of dH2O). Each strain was then diluted to an optical density (OD) of 0.057+/−0.001. Serial dilutions were also performed immediately after diluting all the broths to an OD of 0.057. The density of each strain of bacteria expressed in colony forming units per mL (CFU/mL) following serial dilutions is shown in Table 1.

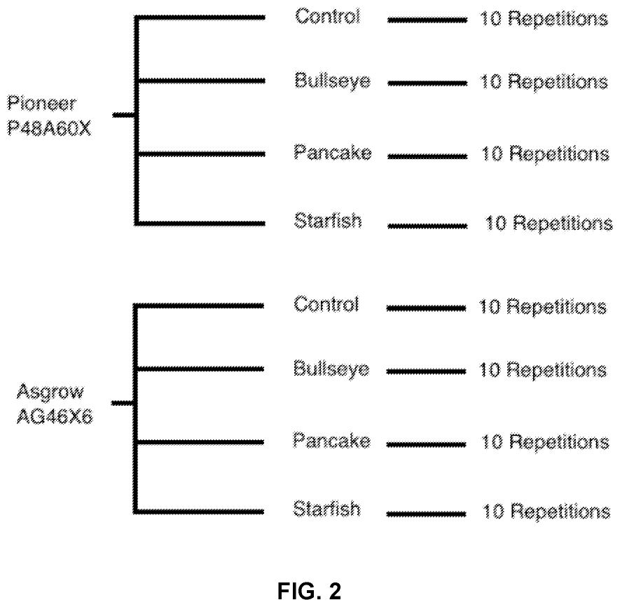

Sixteen days after planting, soybeans were thinned to one plant per pot and the remaining plant in each pot was inoculated with bacteria. Of the 80 total pots, 20 were inoculated with Bullseye, 20 were inoculated with Pancake, and 20 were inoculated with Starfish by pipetting 1 mL of OD 0.057 broth onto the base of each plant. The remaining 20 plants were left uninoculated to serve as controls. Soybeans were in the V2 growth stage at this time. FIG. 2 outlines the treatments and number of treatment repetitions for each soybean cultivar. Immediately following inoculation, pots were randomly distributed throughout the greenhouse in accordance with a completely randomized design. To randomly distribute pots throughout the greenhouse, irrigation tubes were assigned a number 1 to 80; likewise, pots were assigned a number 1 to 80. Once the pots and irrigation tubes were assigned numbers, the random number function in Microsoft Excel (version 16.33; Microsoft Corp., Redmond, WA, USA) was used to randomize the order of the pot of numbers 1-80. The list of irrigation tube numbers was left unrandomized. The first number in the randomized list of pot numbers was then paired with irrigation tube number 1, the second number in the randomized list of pot numbers was paired with the irrigation tube number 2 and so on until all 80 pots were randomly assigned a corresponding irrigation tube.

TABLE 1
Colony forming units per milliliter (CFU/mL) determined by serial
dilution for each strain of bacteria used during inoculation.

| Strain | CFU/mL |
|---|---|
| Bullseye | 4.3 × 10⁷ |
| Pancake | 4.4 × 10⁷ |
| Starfish | 5.4 × 10⁷ |

### Plant Harvest

Plants were harvested at two time points. For the sake of clarity, harvesting in this paper refers to the removal of plants from pots for the purpose of data collection. Five repetitions from each treatment group per cultivar were harvested when plants were in the R2/R3 growth stage (38 days after planting), and the remaining five repetitions from each treatment group per cultivar were harvested when plants were in the R6 growth stage (81 days after planting). The purpose of harvesting at two different time points was twofold. First, it allowed us to determine whether any parameters measured during early stages of flowering served as indicators of growth, yield, and NF during later stages of plant growth. Second, it helped us understand when the effects resulting from inoculants occurred.

A list of the various measurements of growth, yield, NF, and several ancillary variables taken is provided in Table 2. During the first harvest, the following parameters were measured or counted: stomatal conductance, plant height, flower number per plant, the number of trifoliates per plant, length of the second youngest trifoliate, shoot wet and dry mass, root length, root wet and dry mass, number of nodules per plant, nodule wet mass, total plant wet and dry mass, the ratio of shoot to root wet and dry mass, and chlorophyll content. Volumetric water content was measured during the first harvest as well, but this was not considered an indicator of growth, yield, or NF.

The distance between the tip of the center leaf on the trifoliate to the stem was used to determine trifoliate length. Plant height was determined by measuring the stem between the plant-soil interface and the highest node. Plants were removed from the pots and submerged in buckets of water, and the root ball was kneaded to remove soil from the roots. Root length was determined by measuring the distance between the highest lateral root extending out from the stem to the lowest point of the longest root and rounded to the nearest centimeter. Stomatal conductance was determined using a Leaf Porometer Model SC-1 (Decagon Devices Inc., Pullman, WA, USA). The conductance of the bottom side of the leaf was measured the day before harvest. Dry weights were taken after oven-drying samples for a minimum of 72 h at 75° C.-90° C. Samples were considered dry if they became brittle and broke easily when touched.

Plants that did not dry within 72 h were left in the ovens until dry (maximum of 6 days) Chlorophyll content was measured using a SPAD-502 chlorophyll meter (Minolta Co. limited, Japan) the day before harvest. Chlorophyll was assessed by taking the average of three SPAD readings from the center leaf of the second trifoliate from the top of each plant.

During the second harvest, the same parameter measurements collected during the first harvest were repeated. However, stomatal conductance, trifoliate length, trifoliate number, and volumetric water content were excluded during the second harvest. These four parameters were excluded from the second harvest, because harvesting was a time sensitive process and there was not time to collect data on all parameters. Consequently, data on the lowest priority parameters was not collected.

Several additional parameters were also measured during the second harvest. The additional parameters measured or counted during the second harvest include the following: pod wet and dry mass, number of pods per plant, number of seeds per plant, total above ground wet and dry mass, number of auxiliary branches per plant, and seed dry mass. Because NF and leaf nutrient concentration in soybeans peaks in the R5 growth stage then declines (McWilliams et al. 1999), chlorophyll content was measured during the R5 growth stage rather than during the second harvest when plants were in the R6 growth stage. Seed oil and protein content was also measured using a DA 7250 NIR Analyzer (Perten Instruments, Hägersten Sweden). Oil content was not considered an indicator of growth, yield, or NF but is nevertheless an important indicator of seed quality and therefore worthwhile to include.

TABLE 2
List of parameters serving as indicators of soybean
growth, yield, and nitrogen fixation, and additional
ancillary parameters measured during each harvest

| Indicator | | | |
|---|---|---|---|
| Category | Parameter | Harvest 1 | Harvest 2 |
| Nitrogen | SPAD | x | x* |
| Fixation | Nodule number per plant | x | x |
| | Nodule wet mass | x | x |
| | Seed protein content | | x |
| Plant | Plant height | x | x |
| Growth | Trifoliates per plant | x | |
| | Trifoliate length | x | |
| | Root length | x | x |
| | Root wet mass | x | x |
| | Shoot wet mass | x | x |
| | Total above ground wet mass | | x |
| | (pods + shoots) | | |
| | Total wet mass | x | x |
| | Root dry mass | x | x |
| | Shoot dry mass | x | x |
| | Pod dry mass | | x |
| | Total above ground dry mass | | x |
| | (pods + shoots) | | |
| | Total Dry mass | x | x |
| | Shoot/root dry mass ratio | x | x |
| | Auxiliary branches per plant | | x |
| | Shoot/root wet mass ratio | x | x |
| | Shoot/root dry mass ratio | x | x |
| | Stomatal conductance | x | |
| Yield | Flower number per plant | x | x |
| | Pod number per plant | | x |
| | Pod wet mass | | x |
| | Pod dry mass | | x |
| | Seed number per plant | | x |
| | Grain dry mass per plant | | x |
| Ancillary | Volumetric water content | x | |
| Parameters | Seed oil content | | x |

### Iron Solubilization

The ability of each strain of bacteria to produce siderophores was measured by determining whether or not each strain could solubilize Fe using a blue agar chrome azurol S (CAS) assay. Complexed ferric iron causes the CAS agar to appear blue. When blue CAS agar plates are inoculated with siderophore producing bacteria, the siderophores released by the bacteria chelate the Fe causing a color change: a translucent orange halo forms around the bacterial colony.

In the experiment, six CAS agar plates were each divided into four sections, and three of the four sections on each plate were spot inoculated with Bullseye, Pancake, and Starfish respectively. Sterile pipet tips were used to spot inoculate the three sections, and the fourth section remained uninoculated to serve as a control. After inoculation, plates were incubated at 28° C. for two days before assessing plates for color changes.

### Phosphorus Solubilization

The ability of each bacteria strain of interest to solubilize P was determined using National Botanical Research Institute Phosphate (NBRIP) agar media (Nautiyal 1999). The principal behind this method is similar to that described above for detecting Fe solubilization.

When NBRIP plates are inoculated with bacteria that produce phosphate-solubilizing compounds, such as organic acids, a transparent halo develops around the bacterial colony in the otherwise white, opaque media.

In the experiment, six NBRIP plates were divided into four sections each. Three of the sections on each plate were spot inoculated with Bullseye, Pancake, and Starfish using sterile pipet tips. The fourth section served as an uninoculated control. After inoculation, the plates were incubated at 28° C. and assessed for halo formation after two weeks of growth.

Recovery of Bacteria from Nodules

To determine whether or not bacteria used during inoculation could be recovered from soybean nodules, bacteria were extracted from nodules of a representative plant from each treatment group. Nodules obtained from harvested plants were surface sterilized by first submerging 2-5 nodules from a given plant in 0.9% NaCl solution and vortexing on high for 30 s to remove soil particles. The nodules were placed on a sieve containing wire mesh small enough to prevent the nodules from passing through. The nodules in the sieve and the tube in which the nodules were vortexed were then rinsed with deionized (DI) H2O and the nodules were returned to the tube. Nodules were vortexed in NaCl solution and rinsed a total of two times.

After the second rinse, nodules were placed in a clean 2 ml tube, and the tube was filled with 1.65% bleach (NaClO) solution. Nodules were vortexed on high for 30 s then the bleach solution was removed and discarded with a pipet. Nodules were removed and rolled onto sterile filter paper. Nodules were then placed back in the tube and rinsed a second time with the bleach solution. After the second rinse in bleach, nodules were rinsed twice with sterile DI H2O. Tubes containing nodules were filled with sterile DI H2O, vortexed on high for 30 s and the DI H2O was discarded. Nodules were dried by rolling them on sterile filter paper. Nodules were then transferred to a plate containing yeast mannitol agar (YMA) and rolled on the YMA plate for several seconds. The YMA plates were checked after 5 days for growth. If no growth occurred, the outside of nodules was considered sterile.

After sterilizing the outside of nodules, two to three of the sterilized nodules from a given plant were added to 15 mL tubes containing 2 mL of 0.9% NaCl solution. A sterile rod was used to crush the nodules and mix the tubes to suspend nodule bacteria in solution. Plates containing KBC agar were prepared, and 100 μL of NaCl solution containing nodule bacteria was pipetted onto the KBC plates. Colonies that grew on the KBC plates were selected and streaked onto LB agar plates to compare their colony morphology to that of the bacteria used for inoculation.

Transferring the bacteria onto LB agar was necessary, because the colony morphology of inoculant strains differs on different media. If colony morphologies of bacteria extracted from the nodules matched that of inoculant strains, then the 16 s rRNA gene for representative colonies was sequenced to ensure they matched that of the inoculant strains.

### Genome Sequencing and Analysis

For each of the strains used in the study, genomic DNA was isolated using a Puregene DNA Isolation Kit (Cat. No. D-5000A, Minneapolis, MN, USA) following the manufacturer's instructions, and genomic DNA was sent for sequencing to Novogene Corporation Inc. (Sacramento, CA, USA). After sequencing, Illumina reads were trimmed using Trimmomatic (Bolger et al. 2014). Reads were then assembled using ABySS (Jackman et al. 2017), and assemblies were corrected using REAPR (Hunt et al. 2013). Scaffolding was performed using SSpace (Boetzer et al. 2011), and assembly gaps were filled with GapFiller (Boetzer and Pirovano 2012). Assemblies were then annotated using the Prokka pipeline (Seemann 2014). Pseudomonas SBW25 (Genebank accession: NC_012660.1) was used as reference strain, and LINbase (Tian et al. 2019) and an ANI calculator (Rodriguez-R and Konstantinidis 2016) were used to make genome comparisons.

### Statistical Analysis

Multivariate statistical analysis was conducted using PC-ORD version 6 software (MJM Software, Gleneden Beach, OR, USA; McCune and Medfford 2011), and univariate analyses were performed using JMP Pro 15.0.0 software (SAS Institute Inc., Cary, NC, USA). Not all response variables contained normally distributed data or approximately equal variances (Table 24 and Table 25), so two non-parametric multivariate tests were used to analyze the data: multi-response permutation procedures (MRPP; Mielke and Berry 2007) and permutational multivariate analysis of variance (PERMANOVA; Anderson 2001). The data was also analyzed using canonical correspondence analysis (CCA; Braak 1986) and Bray-Curtis ordination (Bray and Curtis 1957) using Euclidean distance. Unlike multivariate analysis of variance (MANOVA), which is used with parametric data, MRPP and PERMANOVA do not allow one to analyze interaction effects. Consequently, the data were broken into pieces and analyzed separately. Ordinates for the two-dimensional solutions were calculated using Polar ordination (similar to PCoA) and Euclidian distance.

The data was divided in two ways. Data were separated by harvest and by cultivar. As a result, four sets of data were created: 1st harvest Asgrow, 1st harvest Pioneer, 2nd harvest Asgrow, and 2nd harvest Pioneer. Each of the four sets of data were analyzed using MRPP and PERMANOVA, and both tests were performed twice, once using unrelativized data and once using relativized data. During the first analysis, raw data from all response variables in each data set were tested, and this raw data is referred to throughout this paper as unrelativized data.

During the second analysis, certain ancillary response variables or those highly correlated with other variables were excluded from each set and the remaining data was relativized. This data is referred to throughout this paper as relativized data.

Relativization standardizes the data to provide a pattern of response whereby measurements associated with each variable are converted to a percent, and the sum total of all the converted variables is equal to 100. To relativize data, each response variable from a given plant is divided by the sum total of all the response variables measured for that plant and multiplied by 100. Each measurement associated with a given response variable from a given plant therefore represent a percent of all the measurements for all the response variables from that plant following the relativization process. Relativization highlights the proportional effect treatments have on different response variables. For example, suppose bacteria caused all response variables to increase, and all the variables increased the same amount relative to all the others. In this case, analysis of relativized data would show bacteria had proportionately equal effects on all variables, and the percentage associated with any given variable would not change in response to the bacteria even though the raw (unrelativized) values all increase. However, if bacteria caused all variables to increase, but the increase in one variable was greater relative to the other variables, then the percentage associated with that variable would increase.

Relativization therefore highlights proportional changes in data in response to treatments while deemphasizing other changes in the raw (unrelativized) data. While other changes in raw data, such as response size, are important, they are generally represented using univariate techniques.

CCA was only used to analyze unrelativized data using constrained multiple regression, while Bray-Curtis ordination was used to analyze both relativized and unrelativized data.

The relativized data did not include certain response variables, because they were either highly correlated with other response variables or they were not central to the main objectives Specifically, root, shoot, and pod wet mass were highly correlated with root, shoot, and pod dry mass respectively. Wet mass is more subject to error than dry mass, because differences in the amount of residual water leftover from cleaning the roots and differences in time the samples spent out of the soil prior to measurement, which leads to different amounts of water loss, can cause variations in measurements. This was expected prior to harvest. Additionally, plants were well watered, so differences in wet mass resulting from drought stress were not expected. Once dry mass data was collected, it was used rather than wet weights.

No dry mass of nodules was taken. Nodules were not dried in order to preserve bacteria living within the nodules by storing them in a −20° C. freezer. Consequently, nodule wet mass was included in the relativized data. Biomass measurements derived from the sum of two or more response variables were also excluded, because these were already accounted for in the model (i.e. total wet and dry mass and total aboveground wet and dry mass). Seed oil content and soil water content were not related to growth, yield, or NF, so these were also excluded from the relativized data. If data were not correlated in the unrelativized data, as predicted, it made sense to remove these data as well. Finally, outliers more than three standard deviations from the mean were excluded as well for the MRPP analyses. The PERMANOVA model does not allow exclusion of outliers, however.

Statistical differences observed following MRPP and PERMANOVA analyses were further explored by performing two-way analysis of variance (ANOVA) tests on response variables included in the multivariate models. Only select variables were analyzed using ANOVA: the highest three vectors with Pearson and Kendall correlation R values of >0.60 or <−0.60 associated with a given axis on each Bray-Curtis plot, or the highest three vectors with inter-set correlation values of >0.60 or <−0.60 associated with a given axis on each CCA plot. If a plot did not have three variables with correlation values>0.60 or <−0.60 on a given Bray-Curtis or CCA plot, only those that did were analyzed further.

Two-way ANOVAs were not appropriate for some response variables, because data corresponding to some response variables were not normally distributed or did not have equal variances (Appendix A: Table 24 Table 25). In such cases, Kruskal-Wallis tests were used to analyze the effect of bacterial treatments for that variable. If results from univariate tests showed significant differences existed, post-hoc Tukey's honestly significant difference analyses were used to determine differences in means of the various treatment combinations. Two-sample t-tests were used to compare means between cultivars, and a significance level (a) of 0.05 was used for all tests.

### Results

### First Harvest: Multivariate Analysis of Growth, Yield, and Nitrogen Fixation

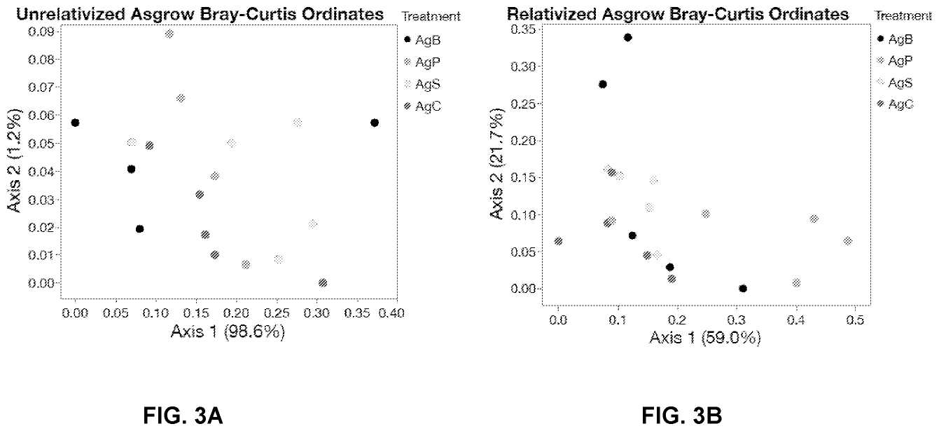

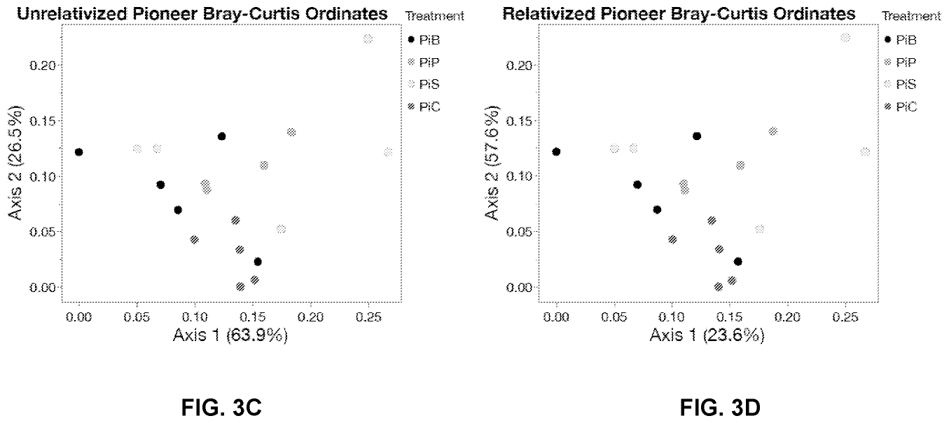

The 1st Harvest, Asgrow, unrelativized data set was the first data set analyzed using MRPP. The main effect from this analysis was significant (p<0.05), which shows differences exist among treatments (Table 3). Pairwise comparisons revealed controls differed from soybeans treated with Pancake and Starfish, but soybeans treated with bacterial inoculants did not differ among each other (Table 4). FIG. 3A shows a Bray-Curtis ordination plot comparing the three bacterial inoculants and the control, and unique clustering patterns of data points representing different treatments indicate differences among the treatments. Furthermore, the variables exerting high levels of influence (R-value or R2>0.6 or <−0.6 on at least one axis) on the model include stomatal conductance, root wet mass, and number of nodules as seen in the table of Pearson and Kendall correlations that corresponds with this data set (Table 7).

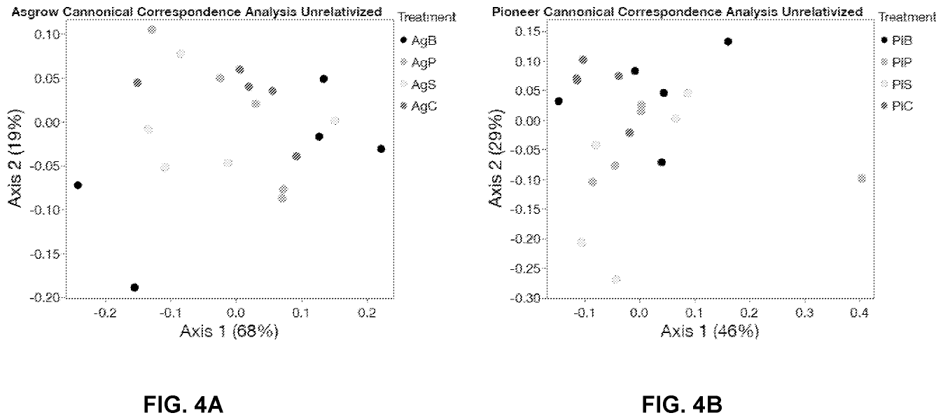

The CCA plot also shows cluster patterns among the different treatment groups, but the CCA plot for unrelativized Asgrow data shows more overlap among the treatments than the Bray-Curtis plot (FIG. 4A). The p-values associated with CCA show no significant differences for MRPP, but they do show differences for PERMANOVA (Table 5 and Table 6). PERMANOVA pairwise comparisons show Pancake and Bullseye differ from the control (Table 6). Variables with high levels of influence (inter-set correlations>0.6 or <−0.6 on at least one axis) on the CCA plot include stomatal conductance and nodules per plant (Table 8).

Results from the Asgrow relativized MRPP analysis of Bray-Curtis ordinates show similar results to that of the unrelativized MRPP analysis: differences (p<0.05) exist among treatments (Table 3). The pairwise comparison results show the control is different from Pancake, but no other comparisons are significant (Table 4). A Bray-Curtis plot displaying the output from the relativized MRPP data is shown in FIG. 3B, and variables resulting in high levels of influence (R-value or R2>0.6 or <−0.6 on at least one axis) over the model include height, trifoliate length, nodule number, nodule mass, and shoot-to-root mass ratio (Table 9).

Results from the Bray-Curtis ordination for Asgrow unrelativized data analyzed using PERMANOVA are generally consistent with those of both MRPP analyses: it shows significant differences exist among treatments (p<0.05; Table 3). Nevertheless, the PERMANOVA also showed greater differences among treatments. It showed lower p-values for the main effect than that of the MRPP analyses, and all pairwise comparisons between bacterial inoculants and controls resulted in significant differences (Table 4). Unlike the results of the MRPP analysis, Starfish also differed from the other two bacteria. Not only were all pairwise comparisons significantly different, all pairwise comparisons resulted in substantially lower p-values than the MRPP pairwise comparison p-values. Results of Asgrow PERMANOVA of relativized data were similar to those of the unrelativized data. Differences existed among treatments (p<0.05; Table 3), and all pairwise comparisons resulted in significant differences (Table 4).

The unrelativized MRPP main effect associated with Bray-Curtis ordination for Pioneer soybeans also showed differences exist among treatments (p<0.05; Table 3). Like the pairwise comparison results from Asgrow, pairwise comparisons for Pioneer showed controls differed from soybeans treated with Pancake and Starfish (Table 4). The Bray-Curtis plot (FIG. 3C) and the CCA plot (FIG. 4B) show the data points representing control treatments occupy a different region of space than data points representing soybeans inoculated with Pancake and Starfish.

This indicates differences exist between the controls and inoculated plants. Because the data points that represent plants inoculated by each of the three bacteria occupy a shared space, there are no differences among inoculation treatments. Additionally, variables with the greatest influence (R-value or R2>0.6 or <−0.6 on at least one axis) on the model used to generate the Bray-Curtis plot include root wet mass, number of nodules, and nodule mass (Table 10).

The Pioneer CCA data resulted in significant differences for both MRPP and PERMANOVA (Table 5). Pairwise comparisons showed the control was different from both Pancake and Starfish for both MRPP and PERMANOVA (Table 6). Variables with the greatest influence (inter-set correlations>0.6 or <−0.6 on at least one axis) over the model used to generate the CCA plot include SPAD, stomatal conductance, and nodules per plant (Table 11) The relativized MRPP main effect for Pioneer Bray-Curtis ordination also resulted in a significant p-value (p<0.05; Table 3). FIG. 3D shows data from the relativized MRPP analysis is indistinguishable from the plot representing data from the unrelativized MRPP analysis (FIG. 3C). Pairwise comparisons following the relativized MRPP analysis show the control only differed from Pancake, but Pancake also differed from Starfish (Table 4). Stomatal conductance, root length, and nodule number had the strongest influence (R-value or R2>0.6 or <−0.6 on at least one axis) over the model used to generate the Bray-Curtis plot (Table 12).

Results from the PERMANOVA analyses of both relativized and unrelativized Pioneer data associated with Bray-Curtis ordination show significant differences among treatments (p<0.05; Table 3), and pairwise comparisons show all bacterial treatments differed from the control (Table 4). Additionally, the main effect of the PERMANOVA analyses of both relativized and unrelativized data for Pioneer soybeans were generally consistent with those of the MRPP analyses, but the PERMANOVA analyses resulted in substantially lower p-values and more differences among the pairwise comparisons.

TABLE 3
First harvest MRPP and PERMANOVA p-values
associated with Bray-Curtis ordination

| | Unrelativized | Relativized | Unrelativized | Relativized |
|---|---|---|---|---|
| Cultivar | MRPP | MRPP | PERMANOVA | PERMANOVA |
| Asgrow | 0.033* | 0.035* | <0.001* | <0.001* |
| Pioneer | 0.022* | 0.027* | 0.002* | <0.001* |

*Indicates p < 0.05

TABLE 4
First harvest Bray-Curtis MRPP and PERMANOVA pairwise comparison
p-values among all four treatment groups for each cultivar

| | Pairwise Comparison P-Values | | | | | | | |
|---|---|---|---|---|---|---|---|---|
| | Unrelativized | | Relativized | | Unrelativized | | Relativized | |
| Treatment | MRPP | | MRPP | | PERMANOVA | | PERMANOVA | |
| Comparison | Asgrow | Pioneer | Asgrow | Pioneer | Asgrow | Pioneer | Asgrow | Pioneer |
| Control vs. | 0.133 | 0.084 | 0.312 | 0.307 | 0.008* | 0.015* | 0.008* | 0.005* |
| Bullseye | | | | | | | | |
| Control vs. | 0.008* | 0.005* | 0.034* | 0.034* | 0.007* | 0.009* | 0.008* | 0.007* |
| Pancake | | | | | | | | |
| Control vs. | 0.016* | 0.015* | 0.454 | 0.543 | 0.010* | 0.007* | 0.008* | 0.006* |
| Starfish | | | | | | | | |
| Bullseye vs. | 0.514 | 0.113 | 0.129 | 0.133 | 0.140 | 0.395 | 0.009* | 0.010* |
| Pancake | | | | | | | | |
| Bullseye vs. | 0.236 | 0.325 | 0.383 | 0.410 | 0.017* | 0.450 | 0.008* | 0.008* |
| Starfish | | | | | | | | |
| Pancake vs. | 0.615 | 0.359 | 0.023* | 0.029* | 0.016* | 0.082 | 0.008* | 0.010* |
| Starfish | | | | | | | | |

*Indicates p < 0.05

TABLE 5
First harvest MRPP and PERMANOVA p-values associated
with canonical correspondence analysis

| Cultivar | MRPP | PERMANOVA |
|---|---|---|
| Asgrow | nd | 0.026* |
| Pioneer | 0.045* | 0.020* |

*Indicates p < 0.05

TABLE 6
First harvest MRPP and PERMANOVA pairwise comparison p-
values associated with canonical correspondence analysis

| | | |
|---|---|---|
| Treatment | MRPP | PERMANOVA |

| Comparison | Asgrow | Pioneer | Asgrow | Pioneer |
|---|---|---|---|---|
| Control vs. | nd | 0.216 | 0.063* | 0.165 |
| Bullseye | | | | |
| Control vs. | nd | 0.037* | 0.035* | 0.028* |
| Pancake | | | | |
| Control vs. | nd | 0.049* | 0.095 | 0.003* |
| Starfish | | | | |
| Bullseye vs. | no | 0.086 | 0.480 | 0.056 |
| Pancake | | | | |
| Bullseye vs. | nd | 0.356 | 0.560 | 0.095 |
| Starfish | | | | |
| Pancake vs. | nd | 0.247 | 0.693 | 0.113 |
| Starfish | | | | |

*Indicates p < 0.05

TABLE 7
Pearson and Kendall correlations with ordination axes derived
from unrelativized data for 1st Harvest, Asgrow soybeans

| | | |
|---|---|---|
| Response | Axis 1 | Axis 2 |

| Variable | R | R² | R | R² |
|---|---|---|---|---|
| SPAD | 0.411 | 0.169 | 0.393 | 0.154 |
| Stomatal | 0.611* | 0.374 | −0.667* | 0.444 |
| Conductance | | | | |
| Height | −0.317 | 0.101 | 0.171 | 0.029 |
| Flowers Per Plant | −0.369 | 0.136 | 0.402 | 0.161 |
| Trifoliates Per | 0.186 | 0.034 | 0.301 | 0.091 |
| Plant | | | | |
| Trifoliate Length | 0.297 | 0.088 | 0.045 | 0.002 |
| Volumetric H₂O | −0.229 | 0.052 | 0.238 | 0.057 |
| Content | | | | |
| Root Length | −0.530 | 0.280 | 0.551 | 0.304 |
| Root Wet Mass | −0.646* | 0.418 | 0.183 | 0.034 |
| Shoot Wet Mass | −0.010 | 0.000 | 0.319 | 0.102 |
| Total Wet Mass | −0.354 | 0.125 | 0.326 | 0.106 |
| Nodules Per Plant | −0.898* | 0.806* | −0.022 | 0.001 |
| Nodules Mass | −0.382 | 0.146 | 0.409 | 0.167 |
| Shoot Dry Mass | −0.048 | 0.002 | 0.308 | 0.095 |
| Root Dry Mass | −0.506 | 0.256 | 0.309 | 0.095 |
| Total Dry Mass | −0.149 | 0.022 | 0.335 | 0.112 |
| Shoot/Root Dry | 0.427 | 0.182 | 0.071 | 0.005 |
| Mass Ratio | | | | |

*Indicates R² or R-value >0.600 or <−0.600

TABLE 8
First harvest Asgrow inter-set correlations associated with
canonical correspondence analysis of unrelativized data

Correlations

| Response Variable | Axis 1 | Axis 2 |
|---|---|---|
| SPAD | −0.473 | −0.262 |
| Stomatal Conductance | −0.442 | 0.745* |
| Height | 0.221 | −0.021 |
| Flowers Per Plant | 0.387 | −0.271 |
| Trifoliates Per Plant | −0.300 | −0.016 |
| Trifoliate Length | −0.284 | 0.205 |
| Root Length | 0.548 | 0.025 |
| Nodules Per Plant | 0.864* | 0.427 |
| Nodule Mass | 0.162 | 0.161 |
| Shoot Dry Mass | 0.043 | −0.066 |
| Root Dry Mass | 0.584 | 0.006 |
| Total Dry Mass | 0.162 | −0.057 |

*Indicates R² or R-value >0.600 or <−0.600

TABLE 9
Pearson and Kendall correlations with ordination axes derived
from relativized data for 1st Harvest, Asgrow soybeans

| Response | Axis 1 | | Axis 2 | |
|---|---|---|---|---|
| Variable | R | R² | R | R² |
| SPAD | −0.527 | 0.278 | 0.453 | 0.205 |
| Stomatal Conductance | −0.316 | 0.100 | 0.197 | 0.039 |
| Height | −0.663* | 0.439 | 0.392 | 0.153 |
| Flowers Per Plant | −0.194 | 0.037 | 0.148 | 0.022 |
| Trifoliates Per Plant | −0.282 | 0.080 | 0.576 | 0.332 |
| Trifoliate Length | −0.639* | 0.409 | 0.556 | 0.309 |
| Root Length | −0.278 | 0.077 | −0.319 | 0.102 |
| Nodules Per Plant | −0.008 | 0.000 | −0.853* | 0.727* |
| Nodule Mass | 0.992* | 0.984* | −0.484 | 0.234 |
| Shoot Dry Mass | −0.201 | 0.040 | 0.588 | 0.345 |
| Root Dry Mass | −0.124 | 0.015 | −0.455 | 0.207 |
| Total Dry Mass | −0.235 | 0.055 | 0.425 | 0.180 |
| Shoot/Root Mass Ratio | −0.433 | 0.188 | 0.881* | 0.776* |

*Indicates R² or R-value >0.600 or <−0.600

TABLE 10
Pearson and Kendall correlations with ordination axes derived
from unrelativized data for 1st Harvest, Pioneer soybeans

| Response | Axis 1 | | Axis 2 | |
|---|---|---|---|---|
| Variable | R | R² | R | R² |
| SPAD | −0.477 | 0.228 | −0.095 | 0.009 |
| Stomatal Conductance | −0.544 | 0.296 | −0.354 | 0.125 |
| Height | −0.314 | 0.099 | −0.230 | 0.053 |
| Flowers Per Plant | −0.360 | 0.130 | −0.024 | 0.001 |
| Trifoliates Per Plant | −0.260 | 0.068 | 0.255 | 0.065 |
| Trifoliate Length | −0.513 | 0.263 | −0.027 | 0.001 |
| Volumetric H₂O Content | −0.093 | 0.009 | −0.532 | 0.283 |
| Root Length | 0.341 | 0.117 | 0.504 | 0.254 |
| Root Wet Mass | −0.617* | 0.380 | −0.247 | 0.061 |
| Shoot Wet Mass | −0.451 | −0.204 | 0.068 | 0.005 |
| Total Wet Mass | −0.586 | 0.343 | −0.056 | 0.003 |
| Nodules Per Plant | −0.842* | 0.708* | 0.170 | 0.029 |
| Nodules Mass | −0.711* | 0.505 | −0.024 | 0.001 |
| Shoot Dry Mass | −0.411 | 0.169 | 0.072 | 0.005 |
| Root Dry Mass | −0.342 | 0.117 | −0.014 | 0.000 |
| Total Dry Mass | −0.408 | 0.166 | 0.055 | 0.003 |
| Shoot/Root Dry Mass Ratio | −0.238 | 0.057 | 0.110 | 0.012 |

*Indicates R² or R-value >0.600 or <−0.600

TABLE 11
First harvest Pioneer inter-set correlations associated with
canonical correspondence analysis of unrelativized data

Correlations

| Response Variable | Axis 1 | Axis 2 |
|---|---|---|
| SPAD | 0.600 | 0.241 |
| Stomatal Conductance | −0.201 | 0.691* |
| Height | 0.285 | 0.275 |
| Flowers Per Plant | 0.365 | 0.223 |
| Trifoliates Per Plant | 0.354 | 0.032 |
| Trifoliate Length | 0.402 | 0.365 |
| Root Length | −0.068 | −0.478 |
| Nodules Per Plant | 0.853* | 0.351 |
| Nodule Mass | 0.565 | 0.512 |
| Shoot Dry Mass | 0.456 | 0.162 |
| Root Dry Mass | 0.215 | 0.299 |
| Total Dry Mass | 0.452 | 0.222 |

*Indicates R² or R-value >0.600 or <−0.600

TABLE 12
Pearson and Kendall correlations with ordination axes derived
from relativized data for 1st Harvest, Pioneer soybeans

| | | |
|---|---|---|
| Response | Axis 1 | Axis 2 |

| Variable | R | R² | R | R² |
|---|---|---|---|---|
| SPAD | 0.485 | 0.236 | 0.133 | 0.018 |
| Stomatal | −0.208 | 0.043 | −0.857* | 0.734* |
| Conductance | | | | |
| Height | 0.437 | 0.191 | 0.005 | 0.000 |
| Flowers Per Plant | 0.055 | 0.003 | 0.035 | 0.001 |
| Trifoliates Per | 0.467 | 0.218 | 0.385 | 0.148 |
| Plant | | | | |
| Trifoliate Length | 0.207 | 0.043 | 0.117 | 0.014 |
| Root Length | 0.769* | 0.591 | 0.664* | 0.441 |
| Nodules Per Plant | −0.888* | 0.789* | 0.319 | 0.102 |
| Nodule Mass | −0.593 | 0.352 | −0.009 | 0.000 |
| Shoot Dry Mass | 0.040 | 0.002 | 0.167 | 0.028 |
| Root Dry Mass | 0.218 | 0.048 | 0.12 | 0.014 |
| Total Dry Mass | 0.079 | 0.006 | 0.164 | 0.027 |
| Shoot/Root Mass | 0.275 | 0.076 | 0.159 | 0.025 |
| Ratio | | | | |

*Indicates R² or R-value >0.600 or <−0.600

### First Harvest Follow-Up Analyses

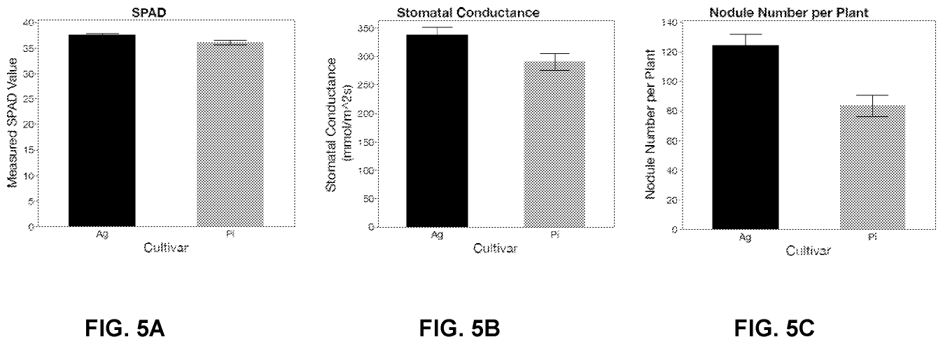

After MRPP and PERMANOVA analyses of data associated with Bray-Curtis ordination and CCA led us to conclude there were differences between bacterial treatments, these differences were investigated further by performing two-way ANOVAs on parametric data and Kruskal-Wallis tests on non-parametric data for each response variable. Limitations of the non-parametric multivariate tests prevented testing of interaction effects between cultivars and bacteria, but the two-way ANOVAs allowed us to test for such interaction effects for each response variable. The following response variables significantly differed (p<0.05) between cultivars: SPAD, stomatal conductance, and nodule number. In all three cases, Asgrow soybeans displayed higher values than Pioneer soybeans (FIG. 5).

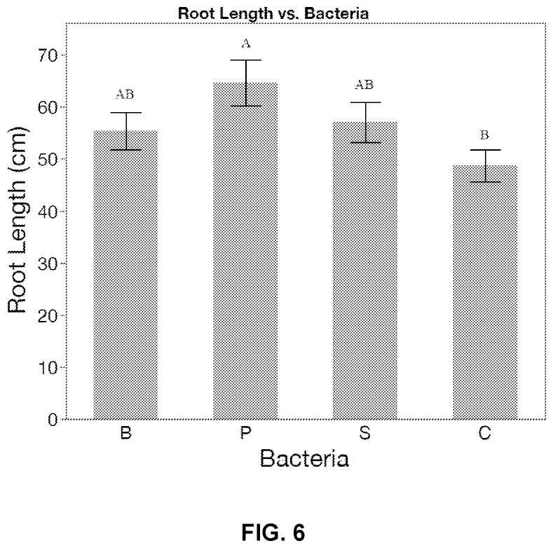

No interaction effects were observed, and only one response variable, root length, differed (p<0.05) among bacterial treatments (FIG. 6). Tukey's HSD revealed Pancake caused plants to produce longer roots than the control group, and roots of plants inoculated with Pancake were 33% longer on average than roots of controls. However, there was no significant difference among plants treated with the three bacterial inoculants, and there was no significant difference between uninoculated plants and those inoculated with Bullseye or Starfish.

### Second Harvest: Multivariate Analysis of Growth, Yield, and Nitrogen Fixation

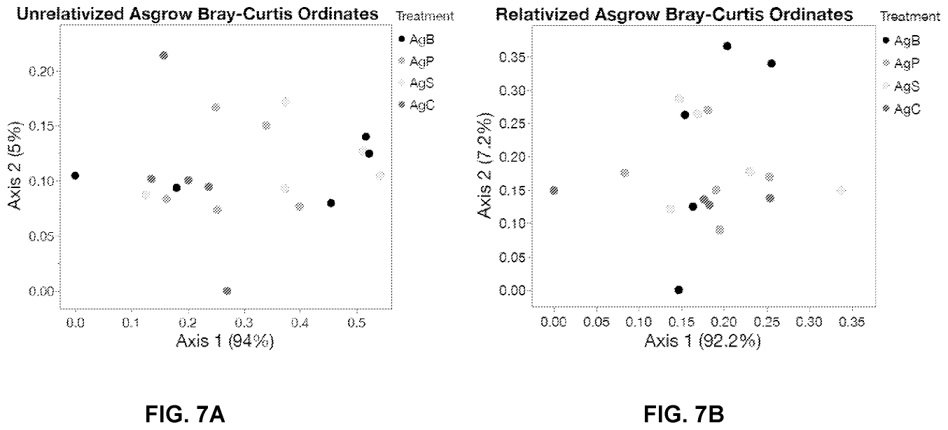

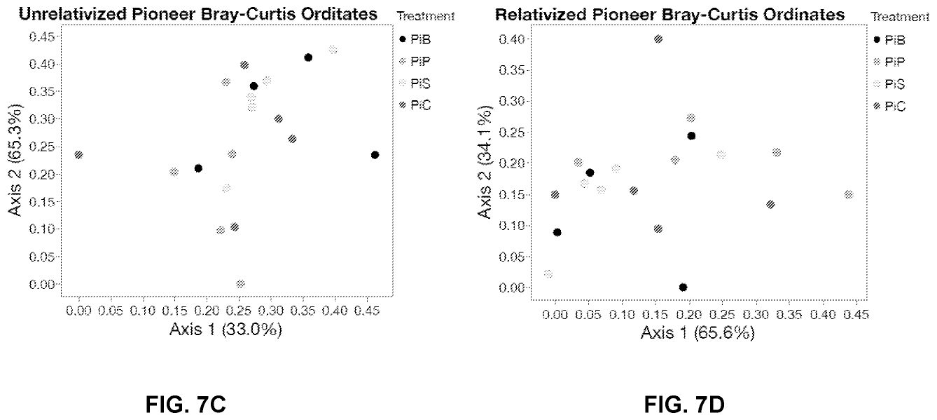

Neither the relativized nor the unrelativized MRPP analyses associated with Bray-Curtis ordination resulted in any significant differences for either cultivar following the second harvest. The PERMANOVA analyses, on the other hand, resulted in significant differences for both soybean cultivars (Table 13). Additionally, pairwise comparisons for both Asgrow and Pioneer show all bacteria were significantly different from the control and from each other based on PERMANOVA results (Table 14). Bray-Curtis plots for unrelativized and relativized data for each cultivar are shown in FIG. 7A-D and corresponding Pearson and Kendall correlations are shown in Table 17, Table 18, Table 19, and Table 20 respectively. None of the Bray-Curtis ordination plots showed separation among the different treatment groups.

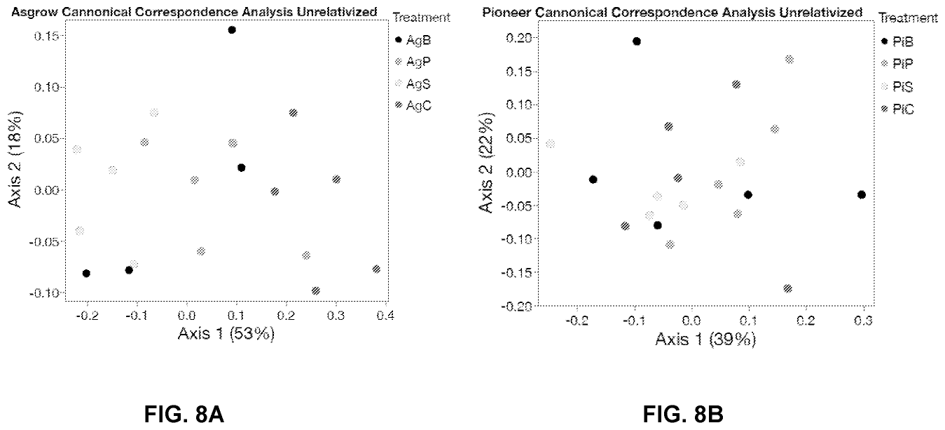

The CCA plot for Asgrow showed separation between points representing controls and those representing the three inoculants, and there appears to be separation between Starfish and Pancake (FIG. 8A). Separation is not apparent in the CCA plot for Pioneer (FIG. 8B). Nevertheless, both the MRPP and PERMANOVA tests show differences exist among both cultivars (Table 15). Pairwise comparisons show controls differed from Pancake and Starfish for both cultivars and both statistical tests (Table 16). Asgrow response variables showing a strong influence over the model (inter-set correlations of >0.6 or <−0.6 for at least one axis) include SPAD, nodules per plant, seeds per plant, seed dry mass, auxiliary branches, and pod dry mass (Table 21). Highly influential variables for Pioneer data include root length and nodules per plant (Table 22).

TABLE 13
Second harvest MRPP and PERMANOVA p-values associated with
Bray-Curtis ordination

| | Unrelativized | Relativized | Unrelativized | Relativized |
|---|---|---|---|---|
| Cultivar | MRPP | MRPP | PERMANOVA | PERMANOVA |
| Asgrow | 0.125 | 0.066 | <0.001* | <0.001* |
| Pioneer | 0.490 | 0.835 | nd^{†} | <0.001* |

*Indicates p < 0.05
^{†}No difference

TABLE 14
Second harvest PERMANOVA pairwise comparison p-values for both
Asgrow and Pioneer Soybeans associated with Bray-Curtis Ordination

| Treatment | Relativized | | Unrelativized | |
|---|---|---|---|---|
| Comparison | Asgrow | Pioneer | Asgrow | Pioneer |
| Control vs. Bullseye | 0.007* | 0.040* | 0.009* | ndt |
| Control vs. Pancake | 0.008* | 0.008* | 0.006* | nd |
| Control vs. Starfish | 0.009* | 0.008* | 0.007* | nd |
| Bullseye vs. Pancake | 0.016* | 0.124 | 0.014* | nd |
| Bullseye vs. Starfish | 0.007* | 0.095 | 0.009* | nd |
| Pancake vs. Starfish | 0.010* | 0.334 | 0.011* | nd |

*Indicates p < 0.05
^{†}No difference

TABLE 15
Second harvest MRPP and PERMANOVA p-values
associated with canonical correspondence analysis

| Cultivar | MRPP | PERMANOVA |
|---|---|---|
| Asgrow | 0.035* | 0.002* |
| Pioneer | 0.010* | <0.001* |

*Indicates p < 0.05

TABLE 16
Second harvest MRPP and PERMANOVA pairwise comparison
p-values associated with canonical correspondence analysis

| | MRPP | | PERMANOVA | |
|---|---|---|---|---|
| Treatment Comparison | Asgrow | Pioneer | Asgrow | Pioneer |
| Control vs. Bullseye | 0.152 | 0.116 | 0.155 | 0.103 |
| Control vs. Pancake | 0.015* | 0.024* | 0.009* | 0.014* |
| Control vs. Starfish | 0.017* | 0.017* | 0.002* | 0.011* |
| Bullseye vs. Pancake | 0.346 | 0.346 | 0.256 | 0.493 |
| Bullseye vs. Starfish | 0.026* | 0.035* | 0.011 | 0.016* |
| Pancake vs. Starfish | 0.237 | 0.029* | 0.047 | 0.015* |

*Indicates p < 0.05

TABLE 17
Pearson and Kendall correlations with ordination axes derived
from unrelativized data for 2nd Harvest, Asgrow soybeans

| Response | Axis 1 | | Axis 2 | |
|---|---|---|---|---|
| Variable | R | R² | R | R² |
| SPAD | 0.084 | 0.007 | 0.533 | 0.284 |
| Height | −0.375 | 0.140 | −0.19 | 0.036 |
| Pod Number | −0.121 | 0.015 | 0.052 | 0.003 |
| Root Length | 0.302 | 0.091 | −0.921* | 0.848* |
| Nodule Number | −0.947* | 0.897* | −0.186 | 0.035 |
| Nodule Mass | −0.463 | 0.214 | −0.262 | 0.069 |
| Shoot Dry Mass | −0.145 | 0.021 | −0.259 | 0.067 |
| Root Dry Mass | −0.389 | 0.151 | −0.363 | 0.132 |
| Seeds Per Plant | 0.032 | 0.001 | 0.172 | 0.030 |
| Seed Dry Mass | −0.361 | 0.130 | 0.033 | 0.001 |
| Auxiliary Branches Per Plant | 0.022 | 0.000 | 0.043 | 0.002 |
| Pod Dry Mass | −0.231 | 0.053 | 0.162 | 0.026 |
| Protein | −0.092 | 0.008 | 0.338 | 0.114 |

*Indicates R² or R-value > 0.600 or < −0.600

TABLE 18
Pearson and Kendall correlations with ordination axes derived
from relativized data for 2nd Harvest, Asgrow soybeans

| Response | Axis 1 | | Axis 2 | |
|---|---|---|---|---|
| Variable | R | R² | R | R² |
| SPAD | −0.802* | 0.643* | −0.421 | 0.177 |
| Height | −0.606* | 0.367 | −0.118 | 0.014 |
| Pods Per Plant | −0.657* | 0.431 | −0.357 | 0.127 |
| Root Length | −0.802* | 0.643* | 0.473 | 0.224 |
| Nodules Per Plant | 0.992* | 0.985* | 0.205 | 0.042 |
| Nodule Mass | 0.0530 | 0.003 | 0.094 | 0.009 |
| Shoot Dry Mass | −0.487 | 0.237 | −0.088 | 0.008 |
| Root Dry Mass | 0.106 | 0.011 | 0.260 | 0.068 |
| Total Dry Mass | −0.731* | 0.534 | −0.320 | 0.102 |
| Shoot/Root Dry Mass | −0.645* | 0.416 | −0.406 | 0.165 |
| Seeds Per Plant | −0.837* | 0.700 | −0.447 | 0.200 |
| Seed Dry Mass | −0.759* | 0.577 | −0.446 | 0.199 |
| Auxiliary Branches Per Plant | −0.412 | 0.170 | −0.146 | 0.021 |
| Pod Dry Mass | −0.829* | 0.687* | −0.515 | 0.265 |
| Above Ground Dry Mass | −0.809* | 0.655* | −0.415 | 0.172 |
| Oil | −0.903* | 0.815* | −0.393 | 0.154 |
| Protein | −0.746* | 0.557 | −0.456 | 0.208 |

*Indicates R² or R-value > 0.600 or < −0.600

TABLE 19
Pearson and Kendall correlations with ordination axes derived
from unrelativized data for 2nd Harvest, Pioneer soybeans

| | Axis 1 | | Axis 2 | |
|---|---|---|---|---|
| Response Variable | R | R² | R | R² |
| SPAD | −0.066 | 0.004 | 0.499 | 0.249 |
| Height | 0.390 | 0.152 | −0.156 | 0.024 |
| Pods Per Plant | 0.140 | 0.019 | 0.145 | 0.021 |
| Root Length | −0.284 | 0.081 | −0.945* | 0.893* |
| Root Wet Mass | −0.286 | 0.082 | −0.471 | 0.222 |
| Shoot Wet Mass | −0.018 | 0.000 | −0.233 | 0.054 |
| Nodule Number | 0.955* | 0.912* | −0.021 | 0.000 |
| Nodule Mass | 0.486 | 0.236 | −0.122 | 0.015 |
| Total Wet Mass | −0.117 | 0.014 | −0.263 | 0.069 |
| Shoot Dry Mass | 0.232 | 0.054 | −0.113 | 0.013 |
| Root Dry Mass | 0.479 | 0.229 | −0.189 | 0.036 |
| Total Dry Mass | 0.362 | 0.131 | 0.026 | 0.001 |
| Shoot/Root Mass Ratio | −0.416 | 0.173 | 0.235 | 0.055 |
| Pod Wet Mass | 0.049 | 0.002 | 0.271 | 0.073 |
| Seeds Per Plant | 0.042 | 0.002 | 0.252 | 0.063 |
| Seed Dry Mass | 0.406 | 0.165 | 0.174 | 0.030 |
| Auxiliary Branches Per Plant | −0.079 | 0.006 | 0.109 | 0.012 |
| Aboveground Wet Mass | 0.012 | 0.000 | −0.019 | 0.000 |
| Pod Dry Mass | 0.279 | 0.078 | 0.285 | 0.081 |
| Aboveground Dry Mass | 0.282 | 0.080 | 0.115 | 0.013 |
| Oil | −0.418 | 0.174 | 0.074 | 0.006 |
| Protein | −0.002 | 0.000 | 0.262 | 0.069 |

*Indicates R² or R-value > 0.600 or < - 0.600

TABLE 20
Pearson and Kendall correlations with ordination axes derived
from relativized data for 2nd Harvest, Pioneer soybeans

| Response | Axis 1 | | Axis 2 | |
|---|---|---|---|---|
| Variable | R | R² | R | R² |
| SPAD | 0.300 | 0.090 | 0.881* | 0.777* |
| Height | 0.480 | 0.231 | 0.767* | 0.588 |
| Pods Per Plant | 0.290 | 0.084 | 0.582 | 0.338 |
| Root Length | 0.766* | 0.586 | −0.477 | 0.227 |
| Nodules Per Plant | −0.855* | 0.730* | −0.671* | 0.451 |
| Nodule Mass | −0.270 | 0.073 | 0.360 | 0.129 |
| Shoot Dry Mass | 0.400 | 0.160 | 0.425 | 0.180 |
| Root Dry Mass | 0.227 | 0.051 | 0.060 | 0.004 |
| Total Dry Mass | 0.332 | 0.110 | 0.670 | 0.449 |
| Shoot/Root Mass | 0.230 | 0.053 | 0.746* | 0.556 |
| Ratio | | | | |
| Seeds Per Plant | 0.056 | 0.003 | 0.855* | 0.732 |
| Seed Dry Mass | 0.250 | 0.063 | 0.816* | 0.666* |
| Auxiliary | 0.056 | 0.003 | 0.433 | 0.188 |
| Branches Per | | | | |
| Plant | | | | |
| Pod Dry Mass | 0.238 | 0.057 | 0.807* | 0.652* |
| Above Ground | 0.331 | 0.109 | 0.734* | 0.539 |
| Dry Mass | | | | |
| Oil | 0.271 | 0.073 | 0.833* | 0.694* |
| Protein | 0.296 | 0.087 | 0.848* | 0.719* |

*Indicates R² or R-value > 0.600 or < −0.600

TABLE 21
Second harvest Asgrow inter-set correlations associated with
canonical correspondence analysis of unrelativized data

| | Correlations | |
|---|---|---|
| Response Variable | Axis 1 | Axis 2 |
| SPAD | 0.298 | 0.638* |
| Height | 0.452 | 0.313 |
| Pods Per Plant | 0.280 | 0.522 |
| Root Length | 0.418 | 0.412 |
| Nodules Per Plant | −0.444 | −0.649* |
| Nodule Mass | 0.041 | 0.054 |
| Shoot Dry Mass | 0.049 | 0.578 |
| Root Dry Mass | 0.035 | 0.122 |
| Seeds Per Plant | 0.286 | 0.627* |
| Seed Dry Mass | 0.297 | 0.621* |
| Auxiliary Branches | −0.191 | 0.613* |
| Pod Dry Mass | 0.332 | 0.626* |
| Protein | 0.365 | 0.538 |

*Indicates R² or R-value > 0.600 or < −0.600

TABLE 22
Second harvest Pioneer inter-set correlations associated with
canonical correspondence analysis of unrelativized data

| | Correlations | |
|---|---|---|
| Response Variable | Axis 1 | Axis 2 |
| SPAD | 0.055 | −0.155 |
| Height | −0.063 | 0.326 |
| Pods Per Plant | −0.300 | 0.155 |
| Root Length | −0.119 | 0.984* |
| Nodules Per Plant | −0.968* | 0.140 |
| Nodule Mass | −0.57 | −0.225 |
| Shoot Dry Mass | 0.004 | 0.373 |
| Root Dry Mass | −0.305 | 0.432 |
| Total Dry Mass | −0.078 | 0.253 |
| Shoot/Root Mass Ratio | 0.429 | −0.277 |
| Seeds Per Plant | −0.268 | −0.336 |
| Seed Dry Mass | 0.002 | −0.03 |
| Auxiliary Branches | −0.106 | −0.137 |
| Pod Dry Mass | −0.033 | 0.015 |
| Above Ground Dry Mass | −0.018 | 0.198 |
| Oil | −0.013 | 0.079 |
| Protein | 0.015 | −0.150 |

*Indicates R² or R-value > 0.600 or < −0.600

### Second Harvest Follow-Up Analyses

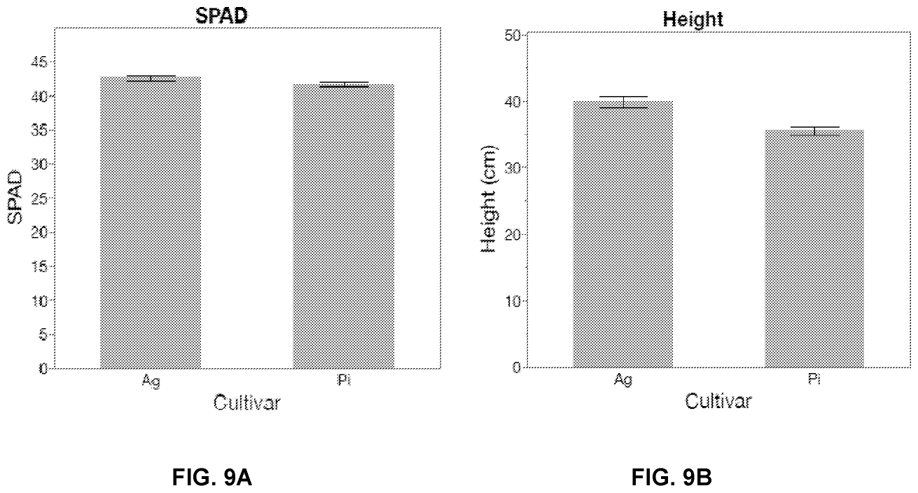

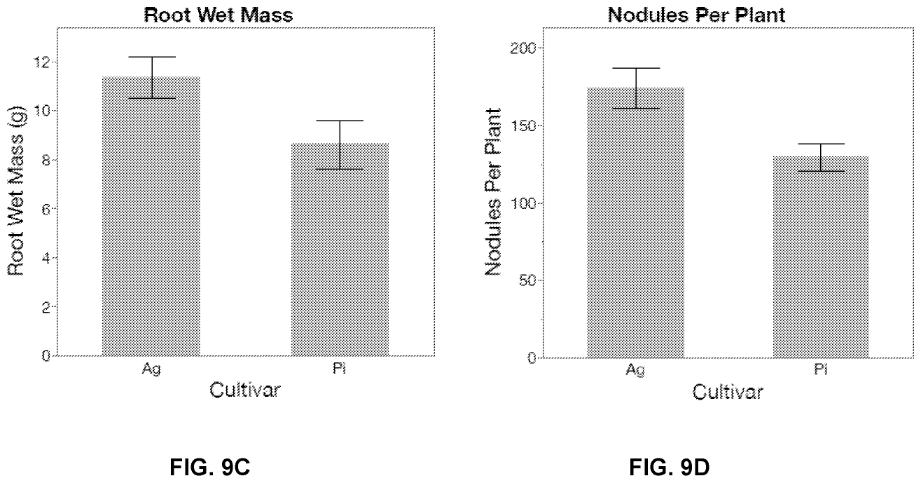

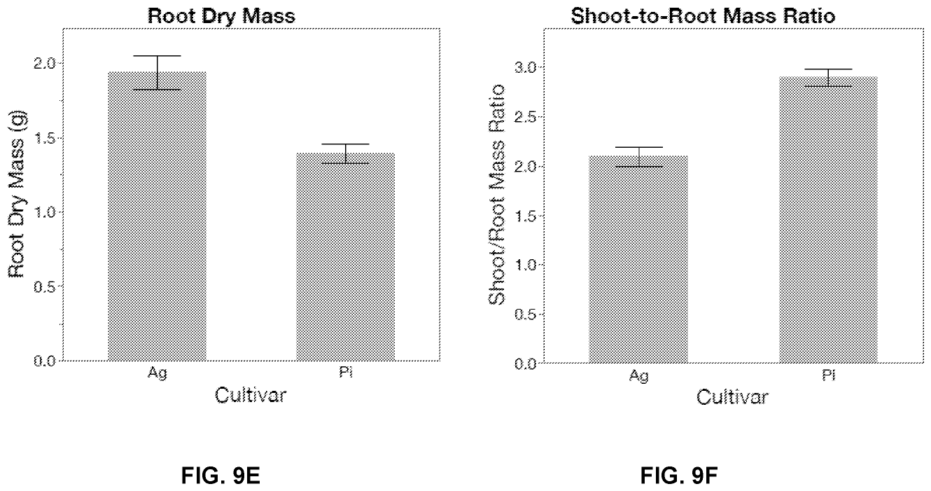

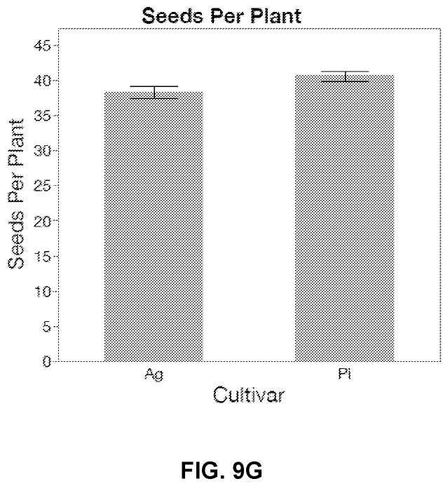

After the second harvest, PERMANOVA analyses associated with Bray-Curtis ordination indicated differences between bacterial treatments likely exist, but MRPP analyses indicated differences likely do not exist. CCA results also showed differences exist among treatments according to both MRPP and PERMANOVA tests. Because PERMANOVA associated with Bray-Curtis ordination, and both MRPP and PERMANOVA associated with CCA showed differences, these differences were investigated further following the same procedures used after the first harvest. Kruskal-Wallis tests for non-parametric data and two-way ANOVAs on parametric data were performed to examine the effect of cultivar and bacteria on each response variable tested. The following response variables significantly differed (p<0.05) between cultivars: SPAD, height, root wet mass, nodules per plant, root dry mass, root-to-shoot mass ratio, and number of seeds per plant. Asgrow plants displayed higher values for all of these variables except shoot-to-root mass ratio and number of seeds per plant (FIG. 9). No significant differences were observed for bacterial treatments.

### Iron and Phosphorus Solubilization

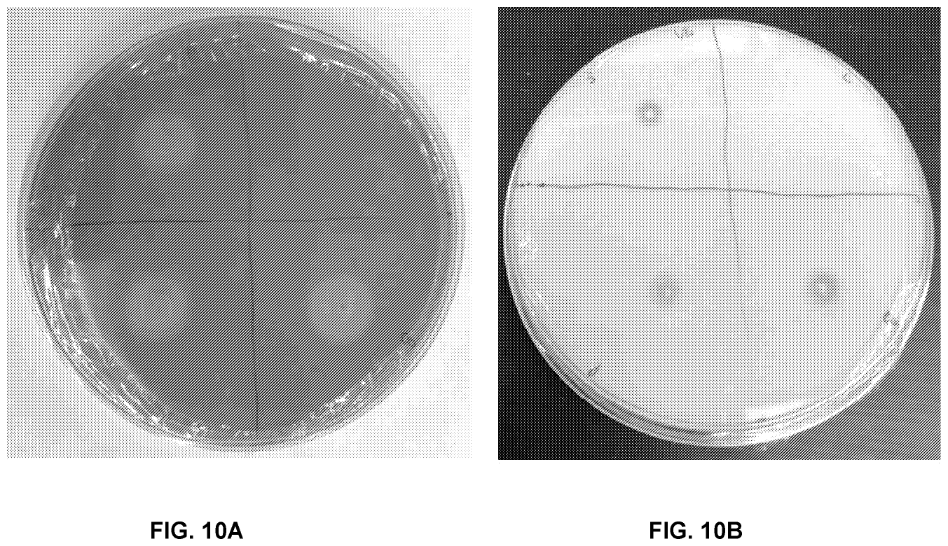

After inoculating Blue CAS agar with each inoculant strain, all bacterial inoculants formed an orange halo around each respective colony on all plates, and a transparent halo formed around colonies grown on all NBRIP plates (FIG. 10). No halos formed in the control quadrants for either type of media. These results indicate all three inoculant strains solubilize both P and Fe.

Recovery of Bacteria from Nodules

After sterilization of the outside of the nodules, crushing the nodules in saline solution, and plating the saline solution on KBC media, inoculant strains of bacteria could not be recovered. Additionally, Bradyrhizobium spp. or any other bacteria could not be cultured on YMA media. These results indicate none of the bacteria within the nodules from either harvest survived. Also, after rolling the outside of the nodules on KBC media then streak-plating colonies onto modified LB media, none of the bacteria from the outside of the nodules appeared identical to the colony morphology of the inoculant bacteria, but some colonies did show morphological similarities. These samples were sent in for 16S rDNA sequencing to determine whether there were any matches with the inoculant strains, but results were inconclusive. Due to logistical challenges arising from the COVID-19 pandemic, these strains could not be resequenced.

### Genome Comparison

According to the ANI calculator, the genomes of the three bacteria used during inoculation were 100.00% identical. Although this value indicates there were no differences among the strains, it displays results out to two decimal places, and small variations (<0.01%) were evident, because the standard deviation for each comparison was greater than zero. Table 23 shows the ANI standard deviation and number of DNA fragments used to for each comparison made.

Output from LINbase showed similar results to those found using the ANI calculator. LINbase is not designed to compare select genomes and only allows users to compare newly uploaded bacterial genomes to those already in the system, so not all the comparisons made using the ANI calculator were able to be made using LINbase. Nevertheless, after the first of the three genomes was added, it served as the most closely related reference strain for the other two.

Consequently, several comparisons between several of the bacteria's genomes were made. Bullseye's genome was the first of the three added, and it was 90.174% similar to the closest genome already uploaded, Pseudomonas fluorescens SBW25. Pancake's genome was uploaded next and was 99.975% similar to that of Bullseye's, and Starfish's genome was 99.980% similar to that of Pancake's.

TABLE 23
Average nucleotide identity standard deviation and
number of DNA fragments used to make comparisons
between each of the three bacteria used for
inoculation. The average nucleotide identity
between all three comparisons made
was 100.00%. Results are based on two-way ANI values.

| Comparison | Standard Deviation | Number of DNA Fragments |
|---|---|---|
| Bullseye-Pancake | 0.11% | 31245 |
| Bullseye-Starfish | 0.04% | 31002 |
| Pancake-Starfish | 0.13% | 31728 |

### Discussion

The results of this study show Pseudomonas spp. isolated from soybeans of one cultivar and used to inoculate other cultivars affected indices of growth and NF after the 1st harvest but results associated with the 2nd harvest were inconclusive. In general, 1st harvest results indicate measures of growth and NF increased in response to Pancake, but there are several caveats to this conclusion. Data associated with increases in growth and NF following inoculation were derived from multivariate analyses, and clustering patterns of Pseudomonas spp. strains differed from clusters representing the uninoculated controls, but only Pancake consistently differed from controls according to both MRPP and PERMANOVA tests associated with Bray-Curtis ordination and CCA. While there were vectors of strong association among response variables, a separate ANOVA analysis did not show significant differences between inoculated plants and controls for any variables, except root length. Notably, inoculated plants generally resulted in greater but not significantly different mean values for these variables (e.g. nodules per plant, nodule mass, plant height, etc.) according to ANOVA results. It was therefore hypothesized that further study would confirm these Pseudomonas spp. strains promote growth and NF.

### Inoculants Affect Indices of Growth and Nitrogen Fixation at 1st Harvest

Because all MRPP and PERMANOVA analyses associated with Bray-Curtis ordination, and all but one of the MRPP and PERMANOVA analyses associated with CCA (MRPP analysis of Asgrow data) showed plants treated with Pancake significantly differed from controls, there is sufficient evidence to conclude Pancake has a significant effect on measures of soybean growth and NF. Only one response variable serving as an indicator of yield, flowers per plant, was measured during the first harvest. While the mean number of flowers on plants treated with Pancake was 14% higher than that of control plants, ANOVA analysis showed this value was not significant. It also did not have a strong influence over the model according to any of the Pearson and Kendall correlations or inter-set correlations in the multivariate analyses. Since this value was not significant, it did not display a high value in the Pearson and Kendall or inter-set correlations, and no other parameters for yield were measured during the 1st harvest.

Follow-up analysis of each parameter measured during the first harvest showed only root length differed between treatments. Specifically, soybeans treated with Pancake had 33% longer roots on average than controls (FIG. 6). This finding is consistent with other research that shows Pseudomonas spp. isolated from mungbean (Noreen et al. 2015) and soybean (Kumawat et al. 2019) nodules promote root growth. Additionally, longer root systems have the potential to improve soybean yield under certain conditions. Hirasawa et al. (1998) measured root length at flowering and found soybeans with longer roots had higher yield at the end of their life cycle then soybeans with shorter root systems when grown during hot summer months under irrigated conditions. They attribute this higher yield to lower resistance to water transport in plants with more developed root systems and suggest such characteristics will likely improve drought tolerance.

Increased rooting depth, which is associated with root length, has been shown to increase drought tolerance in a variety of plant species such as maize (Hund et al. 2009), rice (Li et al. 2005), wheat (Wasson et al. 2012; Li et al. 2019), and soybean (Cortes and Sinclair 1986).

Consequently, if Pancake increases root length, it may increase drought tolerance in soybean. This hypothesis is supported by research that shows adding PGPR to plants can increase root growth, influence root structure, and improve overall drought tolerance (Ngumbi and Kloepper 2016). Production of phytohormones is a common means by which PGPR influence root growth, but environmental factors (Kudoyarova et al. 2019), interactions among different phytohormones, and different concentrations of phytohormones (Weathers et al. 2005) all influence the effect phytohormones have on root growth. One cannot simply conclude that a bacterium that produces phytohormone X will stimulate root growth.

On a related note, Naseem and Bano (2014) inoculated maize with several PGPR, including one Pseudomonas spp., and found the bacteria increased root length, shoot and root mass, relative water content, and other variables under drought stress thus indicating increased drought tolerance. In addition to more developed root systems, the authors attribute improved drought tolerance in inoculated plants to the PGPRs' ability to produce exopolysaccharides (EPS), which help bacteria survive drought stress (Chenu and Roberson 1996; Or et al. 2007).

The authors hypothesize the EPS produced by bacteria likely confers drought resistance to plants the bacteria colonize. This is noteworthy, because the bacteria used in this study produced substances that appeared to be EPS when grown in liquid media and on agar plates.

Consequently, their ability to protect plants from drought stress may be a worthwhile subject of future study.

Although root length was the only response variable to significantly differ among treatments when analyzed using univariate statistics, root length is likely not the only variable causing plants treated with Pancake to differ from controls in the multivariate analysis. Only one of the Pearson and Kendall correlations (Table 12) shows root length had a strong influence over the model (R-value or R2>0.6 or <−0.6 on at least one axis). Why then might multivariate analyses show plants treated with Pancake are significantly different from controls?

Root length was the only response variable to significantly differ among treatments when analyzed using univariate statistics, but the combination of all the response variables, when taken collectively, showed differences based on MRPP and PERMANOVA analyses following the 1st harvest. This finding was interpret as evidence showing Pancake significantly affected parameters for growth and NF without significantly influencing any single response variable measured in isolation, except root length. Additionally, the treatment means for each response variable, when observed separately, indicate Pancake generally increased indices of growth and NF.

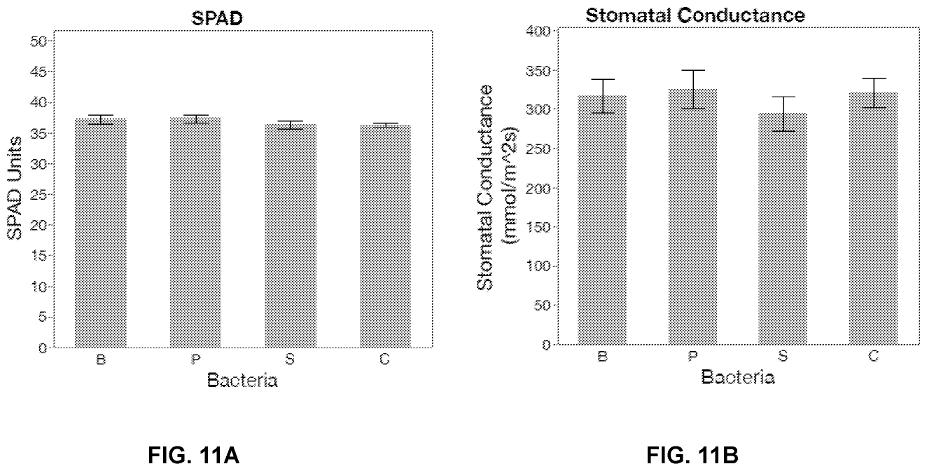

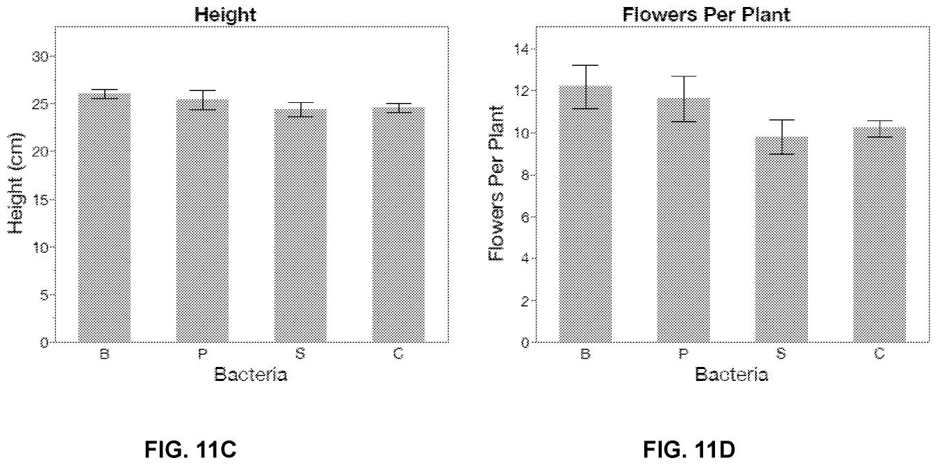

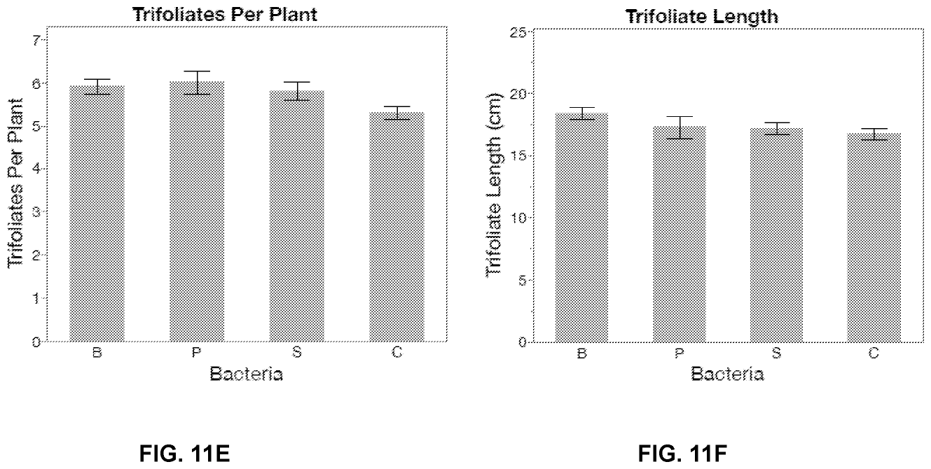

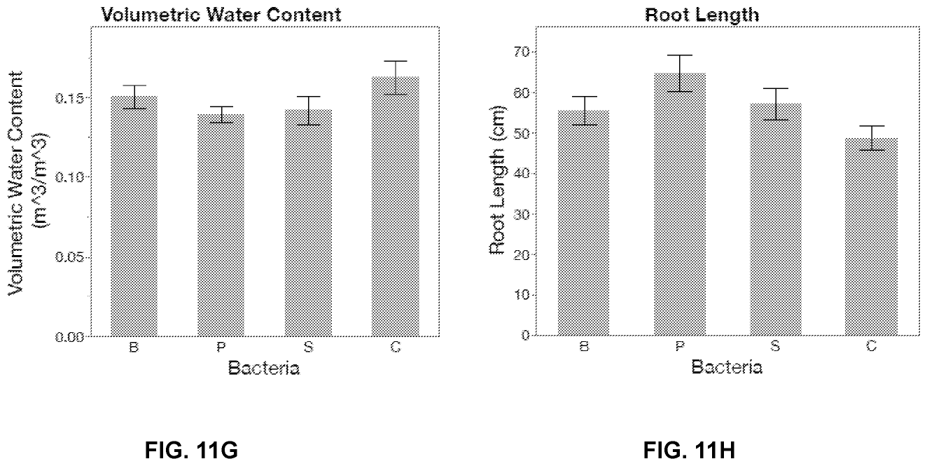

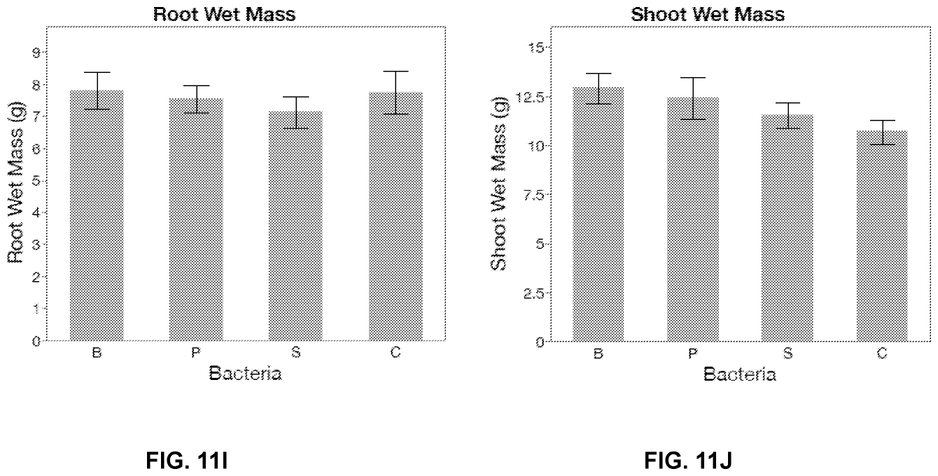

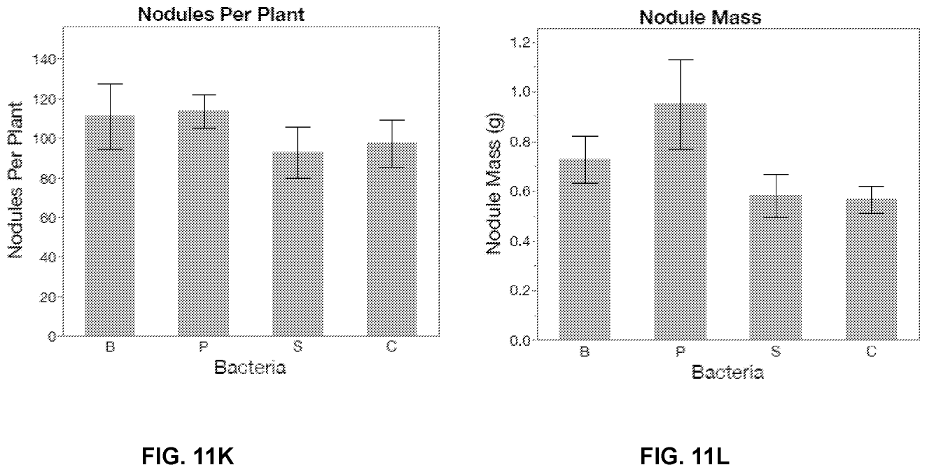

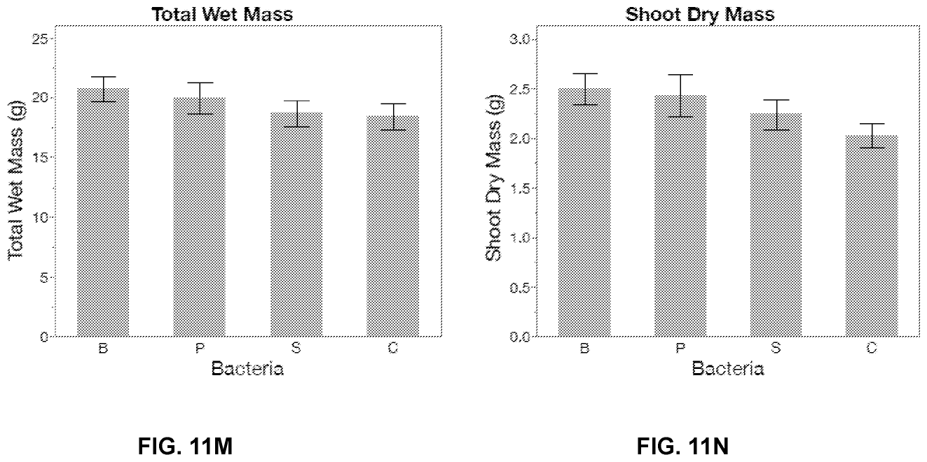

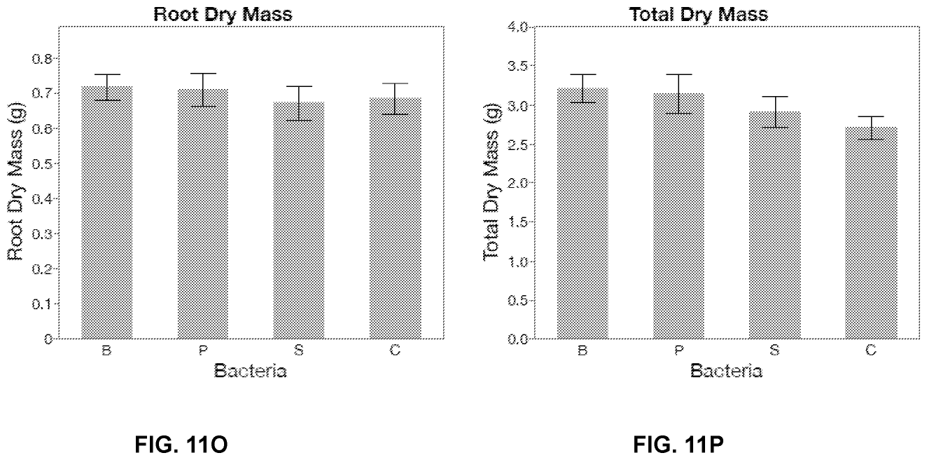

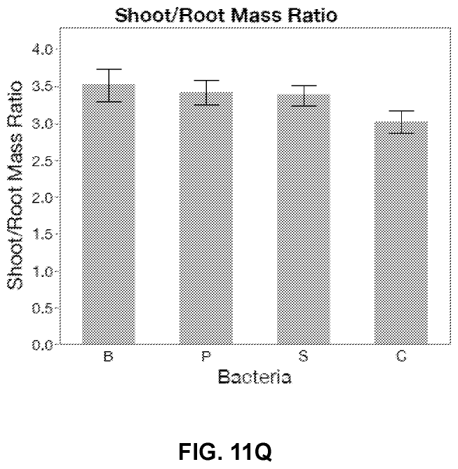

FIGS. 11A-11Q shows plants treated with Pancake displayed higher, though not significantly different, means for every response variable, except volumetric water content and root wet mass, which are less relevant than many of the other response variables. Root wet mass is less relevant than root dry mass, because plants were well watered and residual water remaining after washing roots may have caused minor discrepancies in measurements.

Volumetric water content was one of the ancillary response variables that did not directly measure growth, yield, or NF. When taken in conjunction with the results of the MRPP and PERMANOVA tests, the higher treatment means caused by Pancake generally support the hypothesis that Pancake increases soybean growth and NF during the early stages of soybeans' life cycle.

It is worth noting, nodule number per plant was the only response variable to have a strong influence (Pearson and Kendall R- or R²-value or inter-set correlation value of >0.6 or <−0.6 on at least one axis) on vectors associated with all four 1st harvest Bray-Curtis plots and both canonical correspondence analysis plots (Table 7, Table 8, Table 9, Table 10, Table 11, Table 12).

Pancake caused a 17% average increase in the number of nodules per plant. It also caused a 68% average increase in nodule mass, and nodule mass had a strong influence on variables associated with two Bray-Curtis plots (Table 9 Table 10). Also noteworthy, the Pearson and Kendall nodule mass R-value for Axis 1, Pioneer relativized data was −0.593, which was near the threshold of ±|0.6| used for considering a response variable influential over the model (Table 12).

Nodule mass data was not normally distributed, so a Kruskal-Wallis test was used to assess the difference in means among bacteria. While no significant differences were observed, nodule mass still had a noteworthy p-value of 0.098. These results show further research into the effect of Pancake on nodulation is worth pursuing. Additionally, other studies showing Pseudomonas spp. increase soybean nodulation (Nishijima et al. 1988; Polonenko et al. 1987; Egamberdieva et al. 2017; Kumawat et al. 2019), provides further impetus for studying the effect of Pancake on nodulation.

The most restricting component of symbiotic NF between Rhizobium spp. and legumes is phosphorus (Khan et al. 2007), and P supply influences nodule number and nodule mass in soybeans (Tsvetkova and Georgiev 2003; Miao et al. 2007). More specifically, increases in P lead to increases in nodulation. Because the bacteria used in this study solubilize P, they likely made P more available to the soybeans in this experiment. The increased availability of P may therefore explain, at least in part, the increases in number of nodules and nodule mass in the plants.

Iron availability may have also influenced nodulation in this study. Nodulation is positively correlated with iron availability in legumes (O'Hara et al. 1988; Tang et al. 1990; Jamal et al. 2018), and iron is an essential component in enzymes and proteins that make up the N₂ fixing machinery in diazotrophs (O'Hara 2001). Consequently, iron is essential to the NF process. Additionally, there is evidence that plants take up iron via siderophores produced by Pseudomonas spp. (Wittenberg et al. 1996; Sharma and Johri 2003; Sharma et al. 2003). Since the study indicates the bacteria stimulate nodulation and can produce siderophores, it is reasonable to hypothesize the bacteria increased soybean nodulation by improving accessibility to iron.

Although these bacteria were originally isolated from soybean nodules, bacteria, including Bradyrhizobium spp., were not recovered from the nodules. It is possible freezing and thawing of the nodules killed the bacteria within. Further work should therefore be conducted to confirm whether these bacteria are present within the nodules.

The fact that the Kendall and Pearson correlations showed number of nodules per plant and nodule mass had a strong influence over the model is interesting. Though it was not determined whether the inoculant strains colonized the nodules following inoculation, the results of this study still provide evidence supporting the hypothesis that the bacterium Pancake augments nodulation, and by association, NF. These findings are consistent with research showing Bacillus spp. isolated from nodules of red clover (Trifolium pratense L.) increased nodulation when co-inoculated with Rhizobium sp. (Sturz et al. 1997). While 1st harvest data show Pancake increased soybean growth and NF, there was evidence showing the effects of Bullseye and Starfish on soybean growth and NF as well.

Although, the MRPP and PERMANOVA tests displayed inconsistent and therefore inconclusive results regarding the effects of Bullseye and Starfish on soybean productivity, this study does provide some evidence showing Bullseye and Starfish may affect soybean growth and NF. Both Bray-Curtis PERMANOVA tests resulted in significant differences between controls and Bullseye and between controls and Starfish for both cultivars, and the unrelativized MRPP test resulted in significant differences between controls and Starfish for both cultivars (Table 4).

FIGS. 11A-11Q shows treatment means for all response variables except volumetric water content and stomatal conductance are higher, albeit not significantly different, for plants treated with Bullseye than for controls. Volumetric water was one of the ancillary variables and is not an indicator of growth, yield, or NF, so it is less relevant than other response variables, and stomatal conductance was considered a less reliable measure of growth than other indices, because it is an indirect measurement of growth and may not accurately predict growth. Stomatal conductance is correlated with CO2 assimilation (Ball et al. 1987), but large amounts of photosynthate are dedicated to rhizodeposits (Kuzyakov and Domanski 2000; Butler et al. 2004).

Consequently, large portions of assimilated CO₂ may not be dedicated to growth; hence, stomatal conductance is likely a less reliable indicator of growth than other, more direct measurements. Since 15 of the 17 response variables measured show plants inoculated with Bullseye had higher, though not significantly different, means than controls, it is reasonable to conclude Bullseye increased soybean growth and NF when considering all the response variables collectively and when considering the results of the Bray-Curtis PERMANOVA analyses separately from the MRPP analyses. However, because results of the Bray-Curtis MRPP analyses conflicted with PERMANOVA results, and because among all the CCA results only PERMANOVA analysis of Asgrow data showed Bullseye differed from the control, there is not enough evidence to conclude Bullseye had a significant effect on soybean growth and NF when the totality of evidence presented in this study was consider.

Comparisons of individual treatment means for each response variable is less telling for Starfish than for Bullseye (FIGS. 11A-11Q). Only 10 of the 17 response variables showed plants treated with Starfish had higher treatment means than controls, none of which were significant when analyzed individually. For most response variables, the treatment means for plants inoculated with Starfish were lower than those treated with Bullseye or Pancake. Because multivariate tests show conflicting results, and because plants inoculated with Starfish had lower mean values than other treatments for many response variables, there is insufficient evidence to show Starfish increased soybean growth, yield, or NF. It is reasonable to conclude Starfish had an effect, but the type of effect, positive or negative, is unclear. Additionally, none of the univariate statistics revealed any differences between plants treated with Starfish and controls, so further study into the effects of Starfish on soybean is lower in priority than studying the effects of Bullseye and Pancake.

### Effects of Bacteria at 2nd Harvest are Inconclusive

Results of the MRPP analyses and the PERMANOVA analysis following the 2nd harvest showed conflicting results. No significant differences were observed in either Bray-Curtis MRPP analysis, but the PERMANOVA analysis of both relativized and unrelativized data did show significant differences (Table 13). PERMANOVA analysis of relativized data resulted in significant differences among all pairwise comparisons, and analysis of unrelativized data showed differences exist between controls and Pancake, between controls and Starfish, and between Starfish and the other two bacteria (Table 14). Additionally, all CCA tests showed differences exist among the treatments (Table 15), and pairwise comparisons show controls differed from Pancake and Starfish for both tests for each cultivar (Table 16).

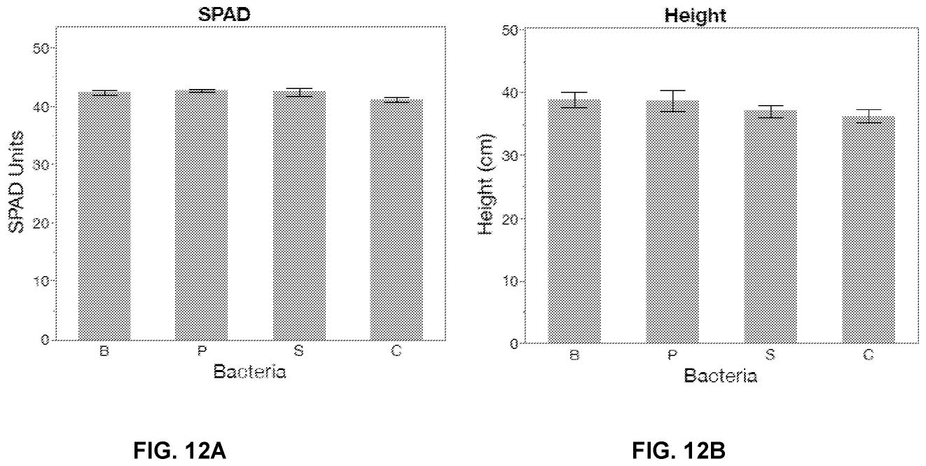

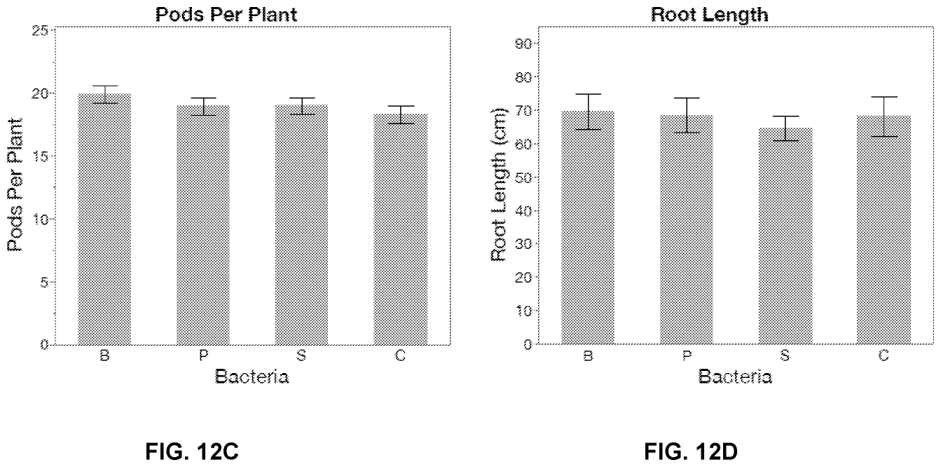

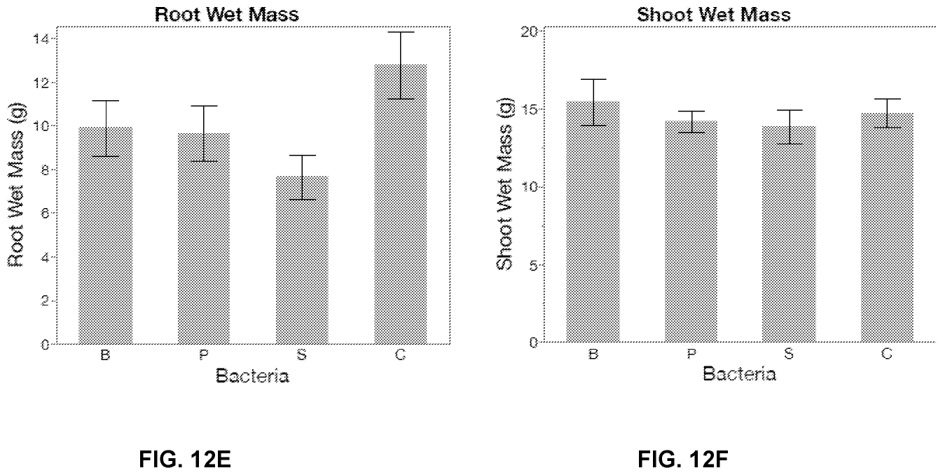

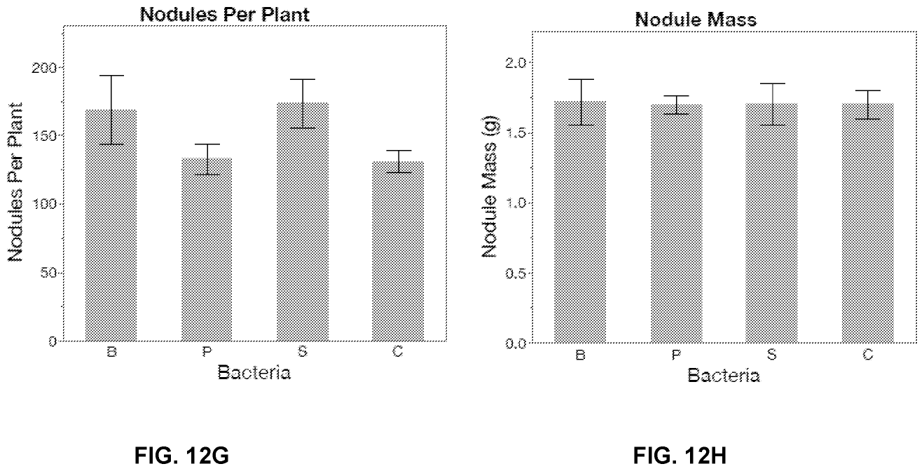

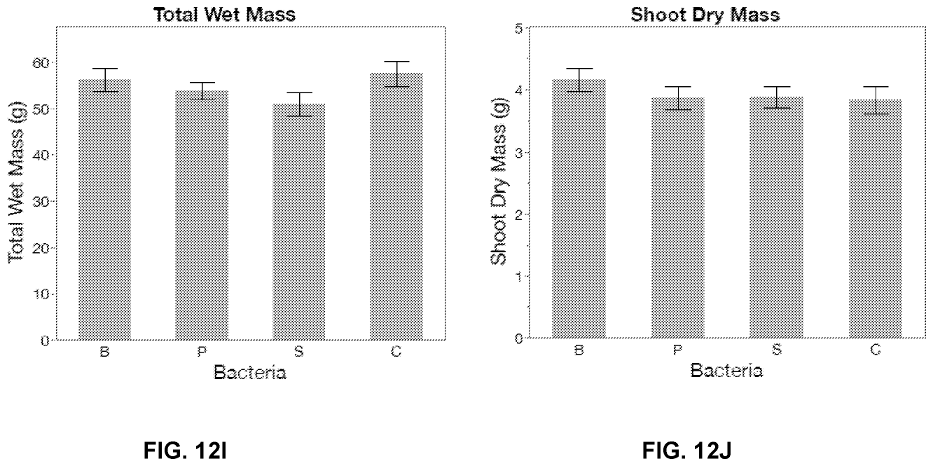

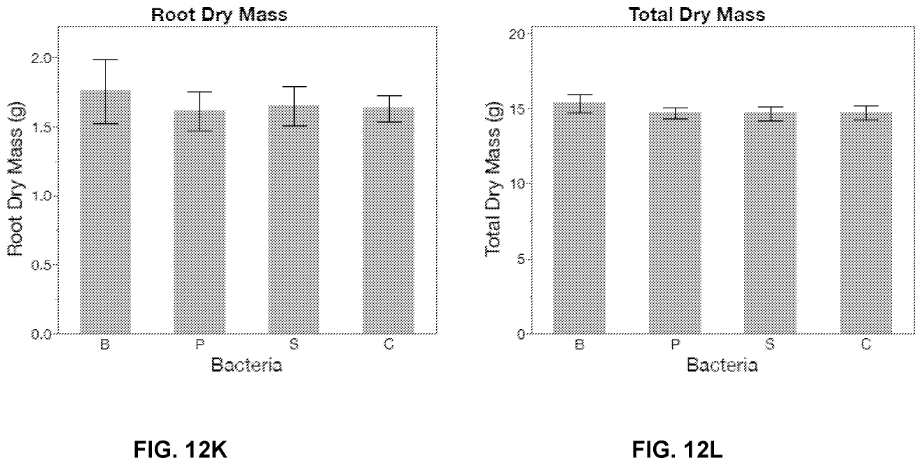

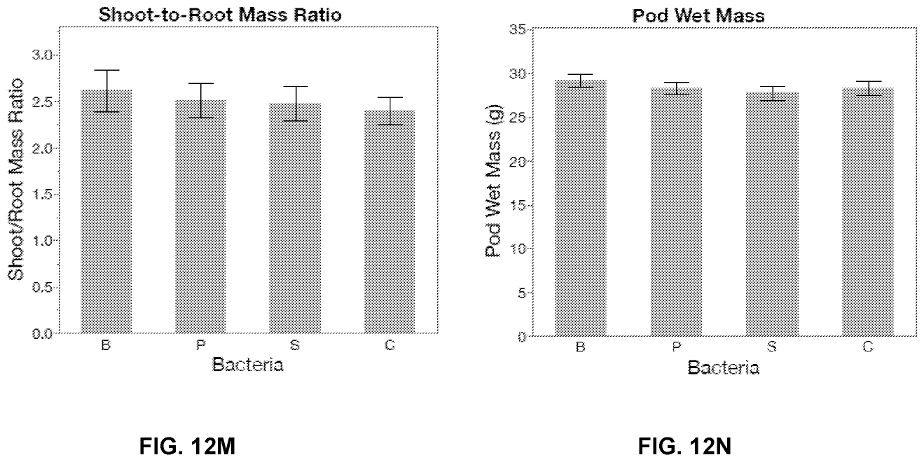

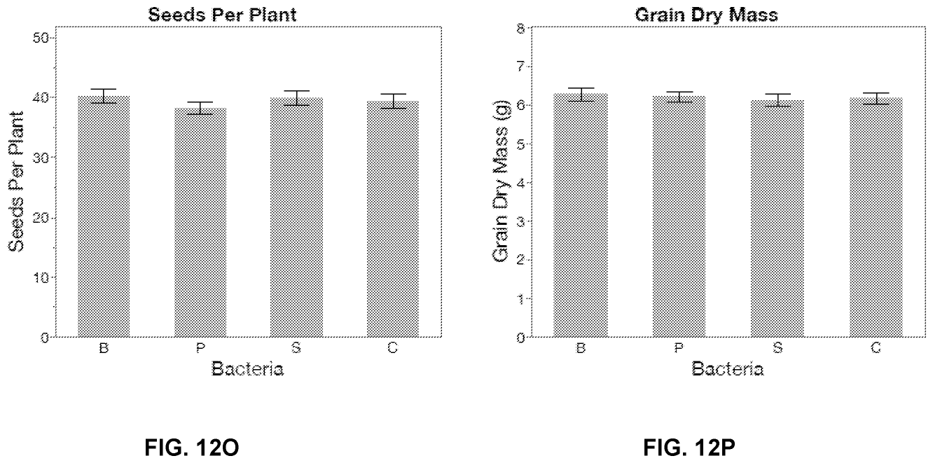

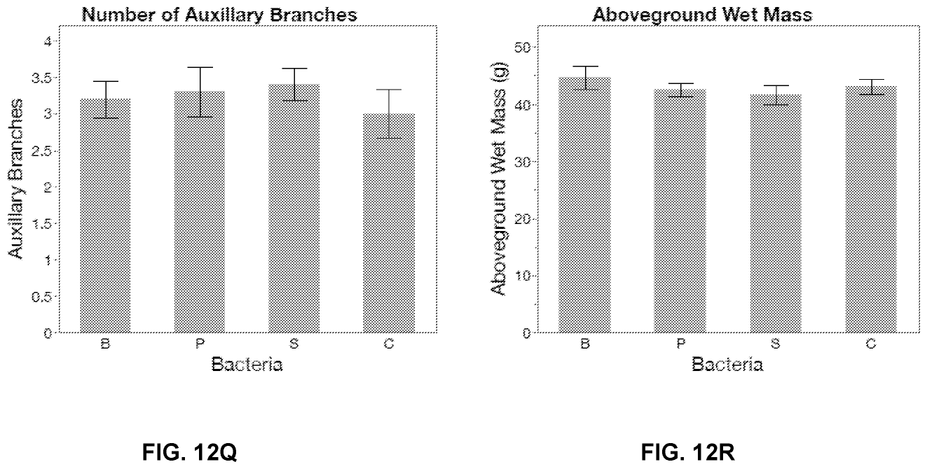

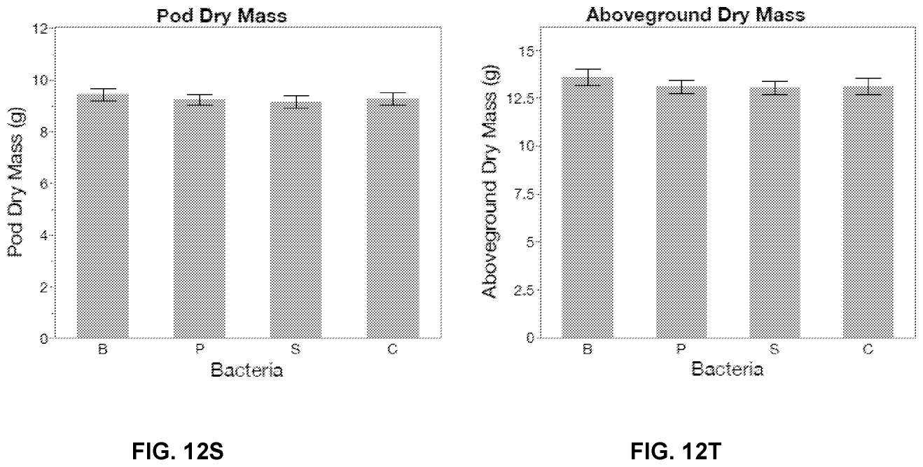

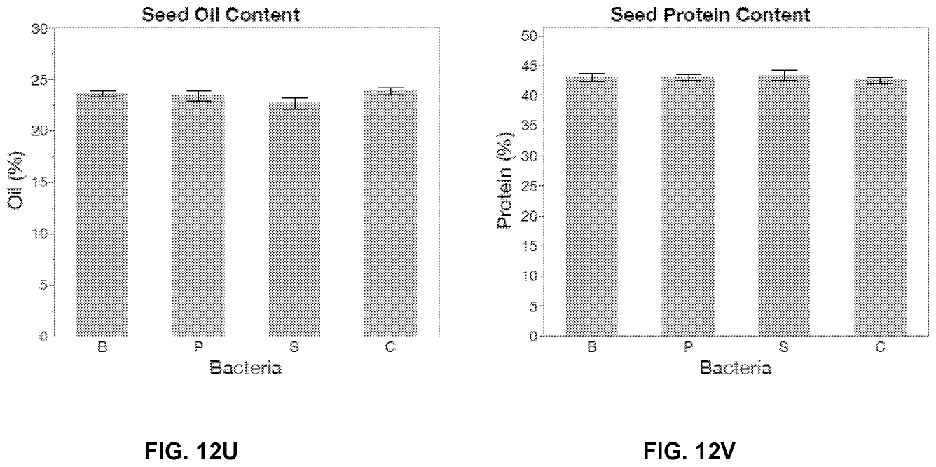

Because no treatment consistently resulted in significant differences across MRPP and PERMANOVA analyses associated with both Bray-Curtis ordination and CCA, and because univariate analyses did not show any significant differences between bacteria and controls, this was not considered enough evidence to draw firm conclusions regarding the effect of the bacteria on soybean growth, yield, or NF at the time of the 2nd harvest. Furthermore, FIG. 12AV shows similar means among treatment groups for most response variables, and the Bray-Curtis plots of 2nd harvest data (FIG. 7) and the Pioneer CCA plot (FIG. 8B) show substantial amounts of overlap among treatment groups. These figures provide visible evidence supporting the conclusion that there are no detectable differences among treatments after the 2nd harvest.

Why are there Different Effects on Soybean Productivity at Different Timepoints?

Results indicate Pancake caused significant increases in soybean growth and NF following the 1st harvest, but it had no effect on 2nd harvest soybeans. If Pancake affects soybeans during an early stage in their life cycle, it is logical, though not imminent, to expect the same trend to continue into later stages. For example, given adequate water and nutrients, if Pancake caused plants to develop biomass at a faster rate than other treatments, the plants should accumulate water, nutrients, CO₂, etc. at a faster rate due to larger root systems, greater leaf surface area for photosynthesis, and other factors. Consequently, the total amount of water, nutrients, etc. accumulated over time should increase, and the total amount of growth, yield, and N2 fixed at the end of the plants' life cycle should increase in response. The plants were well watered, and no signs of nutrient deficiencies were apparent, so water and nutrients likely were not growth limiting factors. Thus, if the bacteria used in the study benefit soybean, the bacteria are expected to increase soybean growth rates and cause greater overall growth, yield, and NF as soybeans approach maturity.

This hypothesis is supported by research that shows high shoot biomass at the beginning of soybean's life is highly correlated with increased growth rate (Vega and Sadras 2003). Also, Malhi et al. (2007) show fertilizer application during early stages of growth is important for legumes to reach maximum biomass and yield. If the bacteria used in the study solubilize nutrients, their effects on plants might be analogous to application of fertilizer: applying PGPR during early stages of growth should increase growth and yield at maturity. Yet this is not what was observed. Why then did Pancake have no effect on 2nd harvest soybeans? The reasons for this response are unclear, but the empirical evidence indicates bacteria have a different effect on soybeans than chemical fertilizers.

The behavior of bacteria applied to soil as biofertilizers is fundamentally different from that of chemical fertilizers, because (except in the case of N2-fixing bacteria) biofertilizers increase solubility of nutrients already in the soil (Mohammadi and Sohrabi 2012), whereas chemical fertilizers add nutrients. One study shows the effects of phosphorus fertilization on plant uptake ranged from 2.6-6.5 years depending on soil type (Eghball et al. 1990). Yet noticeable effects of bacteria applied as biofertilizers are unlikely to last nearly as long. Nutrient limitations, especially carbon, limit microbial growth in soil (Demoling et al. 2007).

Consequently, many bacteria added as inoculants will compete with indigenous microbes for resources and die off until the soil's natural carrying capacity is reached. The remaining inoculant bacteria will eventually reach a state in which they co-exist with indigenous microbial populations at much lower population densities than those present when they were originally applied. In some cases, it's possible they may not survive at all. The effects of biofertilizers may therefore wear off relatively quickly in many cases. This phenomenon in which the inoculant bacteria die off over time might explain why the bacteria used in the study affected soybeans during early stages of growth but not during later stages.

Some runners in a race may jump off the starting blocks and reach their top speed faster than their opponents, but just because they reach their top speed faster than their opponents does not mean they will finish far ahead. Other runners may match a leader's speed shortly after the start. In a similar manner, Pancake may have caused plants to grow significantly faster than other treatments in the days and weeks immediately following inoculation, but the difference between growth rates during this period was not enough to cause significant differences in growth or NF over the course of the plants' lifespan.

If Pancake increased the rate of growth and NF early on in the soybeans' life cycle, it's possible multiple inoculations would maintain this faster rate for a longer period of time. If this faster growth and NF rate were maintained over time, the total growth, yield, and NF would be expected to increase. Research shows repeated inoculations with beneficial microorganisms cause greater increases in plant growth than single inoculations (Azcon 1993).

Another possible reason why the 1st harvest may have resulted in differences but not the 2nd harvest is the constraints caused by the pots. Plants inoculated with Pancake had longer roots than controls after the first harvest, but the pots may have constrained growth as the plants grew larger and could have caused plants to cease growth prematurely. This notion is supported by a meta-analysis that concluded pot size constrains overall plant growth and may change experimental results (Poorter et al. 2012). If pots constrained root growth, such constraints may have counteracted the effects of the bacteria on root growth, which could have also reduced growth and other functions elsewhere in the plant. More specifically, constrained roots can reduce plants' ability to take up nutrients from the soil, which in turn can suppress growth (Poorter et al. 2012). Also, constraints on roots may cause plants to release signals suppressing aboveground growth (Young et al. 1997). Future studies should therefore test the effects of Pancake on plants under conditions where root growth is less constrained.

The soybeans were planted late in the season and the photoperiod during this time appeared to cause plants to mature early thus preventing them from reaching their maximum size. Both cultivars were part of the same maturity group (group 4), so differences between cultivars were likely not attributed to different responses in photoperiod. Nevertheless, it is possible the effects of the different bacterial treatments may have been deemphasized since the plants matured more quickly. If the plants matured more quickly, they would not reach their maximum size and yield. If the bacteria increased the soybeans' maximum growth potential, but the soybeans were unable to reach their maximum potential due to their response to the photoperiod late in the season, the effect of the bacteria on soybean growth may be less apparent.

Finally, it is worth noting the natural variability of the various response variables measured made it difficult to detect differences with the sample sizes used in this study. A power analysis shows five repetitions of each bacteria-cultivar treatment combination per harvest and ten repetitions for each bacterial treatment per harvest were not enough to detect differences for most response variables when analyzed individually, especially during the 2nd harvest (Table 26 and Table 27).

There was a large variation in the number of repetitions needed to detect differences in different response variables, and the second harvest required larger sample sizes to detect differences than the first harvest. When analyzing second harvest data, a sample size of 10, 12, and 15 plants per bacteria-cultivar combination would have allowed us to detect differences among bacterial-cultivar treatment interactions in 63.6%, 72.7% and 86.4% of response variables respectively when effects were present. Larger samples sizes than these would be needed to detect differences among bacterial treatments alone, and such large sample sizes would be impractical to implement.

### Relevance of Findings

The strains used in this study were taken from the cultivar 5002T, one of the cultivars used by (Sharaf et al. 2019), but they were not among the most abundant Pseudomonas present in nodules. Among Pseudomonas spp. found in the preceding study by Sharaf et al. (2019), the bacteria in the study ranked 41st out of 412 OTUs and made up 7% of the total Pseudomonas spp. population (unpublished data). They only made up approximately 0.4% of the total bacterial population, which contained 4068 OTUs. OTUs were based on the 341F-785R region of the 16S rDNA gene.

The findings of this study are important because they show non-Bradyrhizobium spp. bacteria isolated from soybean nodules affect soybean productivity and likely benefit the plant, which provide impetus for further investigation into the possible benefits of non-Bradyrhizobium spp. nodule endophytes. If the bacteria in the study were relatively low in abundance compared to other Pseudomonas spp. in the nodule, but they still benefit the plant, other Pseudomonas spp. found in greater abundance may provide even greater benefits to soybeans than those used in this study. This study provides promising evidence that such nodule endophytes have the potential to be developed into biofertilizers.

### Conclusion

Results of the multivariate analysis show Pseudomonas spp. isolated from soybean nodules of one cultivar and used to inoculate other cultivars significantly affected indices of growth and NF during early stages of the soybeans' lifecycle but affects during later stages were not detected. When indices of growth, yield, and NF were analyzed individually, only root length significantly increased in response to bacterial inoculation, but the means of most indices were generally higher in inoculated plants than in controls. Taken collectively, these results generally support the hypothesis that Pseudomonas spp. isolated from soybean nodules increase soybean growth and NF, but further testing is needed to corroborate these findings before drawing firm conclusions. These findings are important, because they show non-Bradyrhizobium spp. bacteria within nodules have the potential to increase soybean productivity. Future work should therefore focus on culturing similar nodule bacteria and identifying strains with the most beneficial effects on soybean growth, yield, NF, drought, and other important traits.

### Variance and Distribution

The variance of cultivar and bacteria data was assessed separately for each harvest. Because data did not have equal variances and normal distributions for all response variables, non-parametric multivariate tests, PERMANOVA and MRPP, were used to analyze the data. First harvest variance also includes concatenated cultivar-bacteria data, because interaction effects were tested following multivariate tests. If concatenated data showed equal variances, and if data were normally distributed, two-way ANOVAs were used to assess the data. If variances of concatenated data were not equal, or if distributions were not normal, Kruskal-Wallis tests were used to test for differences among bacterial treatments. JMP version 15 software was used to test normality and variance. Anderson-Darling's test was used to assess normality, and Levene's test was used to assess variance.

TABLE 24
First harvest distribution and variance for cultivar, bacteria, and
concatenated bacteria and cultivar data for each response variable.
Anderson-Darling goodness-of-fit p-values > 0.05 indicate normal
distribution and Levene p-values > 0.05 indicate equal variance.

| | | | | Concatenated |
|---|---|---|---|---|
| | | | | Cultivar- |
| | Distribution: | Cultivar | Bacteria | Bacteria |
| | Anderson- | Variance: | Variance: | Variance: |
| Response | Darling p- | Levene p- | Levene p- | Levene p- |
| variable | values | values | values | values |
| SPAD | 0.950 | 0.411 | 0.358 | 0.382 |
| Stomatal | 0.074 | 0.312 | 0.882 | 0.780 |
| Conductance | | | | |
| Height | 0.838 | 0.512 | 0.029* | 0.208 |
| Flowers Per | 0.003* | 0.569 | 0.226 | 0.044* |
| Plant | | | | |
| Trifoliates | <0.001* | 0.015* | 0.576 | 0.015* |
| Per Plant | | | | |
| Trifoliate | 0.081 | 0.309 | 0.723 | 0.216 |
| Length | | | | |
| Volumetric | 0.436 | 0.835 | 0.146 | 0.474 |
| Water | | | | |
| Content | | | | |
| Root Length | 0.142 | 0.019* | 0.777 | 0.245 |
| Root Wet | 0.479 | 0.727 | 0.282 | 0.374 |
| Mass | | | | |
| Shoot Wet | 0.554 | 0.592 | 0.543 | 0.325 |
| Mass | | | | |
| Nodules Per | 0.300 | 0.633 | 0.092 | 0.001* |
| Plant | | | | |
| Nodule Mass | <0.001* | 0.105 | 0.007* | 0.032* |
| Total Wet | 0.768 | 0.972 | 0.993 | 0.429 |
| Mass | | | | |
| Shoot Dry | 0.060 | 0.316 | 0.591 | 0.500 |
| Mass | | | | |
| Root Dry | 0.482 | 0.232 | 0.878 | 0.537 |
| Mass | | | | |
| Total Dry | 0.149 | 0.257 | 0.799 | 0.598 |
| Mass | | | | |
| Shoot/Root | 0.002* | 0.611 | 0.875 | 0.231 |
| Mass Ratio | | | | |

TABLE 25
Second harvest distribution and variance for cultivar and
bacteria for each response variable. Anderson-Darling
goodness-of-fit p-values > 0.05 indicate normal
distribution and Levene p-values > 0.05 indicate equal variance.

| | | Cultivar | Bacteria |
|---|---|---|---|
| | Distribution: | Variance: | Variance: |
| Response | Anderson- | Levene | Levene |
| variable | Darling p-values | p-values | p-values |
| SPAD | 0.826 | 0.940 | 0.007* |
| Height | 0.166 | 0.475 | 0.862 |
| Pods Per Plant | 0.068 | 0.134 | 0.996 |
| Root Length | 0.051 | 0.057 | 0.569 |
| Root Wet Mass | 0.603 | 0.146 | 0.817 |
| Shoot Wet | 0.605 | 0.693 | 0.435 |
| Mass | | | |
| Nodules Per | 0.001* | 0.069 | 0.006* |
| Plant | | | |
| Nodule Mass | 0.863 | 0.375 | 0.004* |
| Total Wet Mass | 0.646 | 0.964 | 0.879 |
| Shoot Dry Mass | 0.240 | 0.622 | 0.836 |
| Root Dry Mass | 0.051 | 0.156 | 0.106 |
| Total Dry Mass | 0.239 | 0.776 | 0.668 |
| Shoot/Root | 0.180 | 0.906 | 0.531 |
| Mass Ratio | | | |
| Pod Wet Mass | 0.050 | 0.762 | 0.725 |
| Seeds Per Plant | 0.495 | 0.482 | 0.836 |
| Seed Dry Mass | 0.305 | 0.916 | 0.570 |
| Auxiliary | <0.001* | 0.399 | 0.305 |
| Branches | | | |
| Aboveground | 0.471 | 0.971 | 0.270 |
| Wet Mass | | | |
| Pod Dry Mass | 0.464 | 0.577 | 0.702 |
| Aboveground | 0.255 | 0.459 | 0.864 |
| Dry Mass | | | |
| Oil | 0.068 | 0.093 | 0.315 |
| Protein | 0.567 | 0.257 | 0.284 |

### Power Analysis

A power analysis for each harvest was performed using JMP's statistical power calculator to determine the sample size needed to identify differences among the treatment groups. Desired power was set to 0.8, significance was set to 0.05, and the mean value of each treatment group for each response variable found in the study was added to the statistical power calculator. This allows us to determine the sample sizes that likely would have been necessary to detect differences in the study. Two separate calculations were made for each response variable: one for bacterial treatments in which the means of all four bacterial treatments were entered, and one for bacteria-cultivar treatment combinations in which the eight treatment combination means were entered. The combined standard deviation of the Asgrow and Pioneer control groups was used in the standard deviation input in both calculations. The calculator output provided a sample size, and that sample size was divided by the number of treatment groups (four bacterial treatments and eight bacteria-cultivar combinations) to determine the recommended sample size per treatment. For reference, in this study a sample size of ten per bacterial treatment and five per bacteria-cultivar treatment combination in each harvest was used.

TABLE 26
First Harvest recommended sample size for interaction effect and
bacteria treatment. The associated standard deviation of combined
Asgrow and Pioneer control groups needed to determine differences
among treatments for each response variable is also shown.

| | Control | Recommended Sample | |
|---|---|---|---|
| | Standard | Size Per Treatment | |
| Response Variable | Deviation | Bacteria-Cultivar | Bacteria |
| SPAD | 1.08 | 3.4 | 13.8 |
| Stomatal | 54.8 | 7.0 | 60.0 |
| Conductance | | | |
| Height | 1.47 | 8.1 | 15.0 |
| Flowers Per Plant | 1.17 | 2.9 | 5.0 |
| Trifoliates Per Plant | 0.46 | 4.5 | 9.0 |
| Trifoliate Length | 1.36 | 7.0 | 13.7 |
| Volumetric Water | 0.03 | 10.5 | 36.8 |
| Content | | | |
| Root Length | 9.34 | 3.8 | 8.5 |
| Root Wet Mass | 1.99 | 27.3 | 151.3 |
| Shoot Wet Mass | 1.85 | 7.0 | 14.3 |
| Nodules Per Plant | 36.05 | 5.4 | 46.8 |
| Nodules Mass | 0.16 | 2.0 | 4.0 |
| Total Wet Mass | 3.30 | 3.1 | 34.8 |
| Shoot Dry Mass | 0.36 | 4.8 | 10.8 |
| Root Dry Mass | 0.13 | 29.3 | 109 |
| Total Dry Mass | 0.46 | 7.5 | 16.8 |

| | | |
|---|---|---|
| Average Recommended Sample | 8.4 | 34.4 |
| Size Per Treatment | | |
| Median Recommended Sample | 6.2 | 14.7 |
| Size Per Treatment | | |
| Standard Deviation | 7.9 | 40.0 |

TABLE 27
Second Harvest recommended sample size for interaction effect and
bacteria treatment. The associated standard deviation of combined
Asgrow and Pioneer control groups needed to determine differences
among treatments for each response variable is also shown.

| | Control | Recommended Sample | |
|---|---|---|---|
| | Standard | Size Per Treatment | |
| Response Variable | Deviation | Bacteria-Cultivar | Bacteria |
| SPAD | 1.31 | 5.0 | 13.3 |
| Root Length | 17.87 | 73.4 | 232.5 |
| Root Wet Mass | 4.56 | 8.0 | 17.5 |
| Shoot Wet Mass | 2.73 | 6.0 | 64.0 |
| Nodules Per Plant | 24.19 | 2.1 | 6.0 |
| Nodule Mass | 0.30 | 11.8 | 6960.0 |
| Total Wet Mass | 7.95 | 8.8 | 28.3 |
| Pod Wet Mass | 2.49 | 14.4 | 60.0 |
| Pods Per Plant | 8.80 | 201 | 646.8 |
| Seeds Per Plant | 3.64 | 6.9 | 63.3 |
| Seed Dry Mass | 0.43 | 12.3 | 183.3 |
| Auxiliary Branches | 1.00 | 25.2 | 125.8 |
| Plant Height | 3.18 | 3.6 | 22.5 |
| Total Aboveground | 4.23 | 7.3 | 42.3 |
| Wet Mass | | | |
| Shoot Dry Mass | 0.66 | 11.8 | 70.0 |
| Root Dry Mass | 0.28 | 2.6 | 75.8 |
| Total Dry Mass | 1.40 | 8.4 | 80.3 |
| Pod Dry Mass | 0.72 | 2.0 | 86.0 |
| Total Aboveground | 1.24 | 12.5 | 77.3 |
| Dry Mass | | | |
| Shoot/Root Mass | 0.44 | 3.1 | 92.5 |
| Ratio | | | |
| Oil | 1.04 | 3.0 | 13.8 |
| Protein | 1.58 | 9.9 | 68.0 |

| | | |
|---|---|---|
| Average Recommended Sample | 20.0 | 410.4 |
| Size Per Treatment | | |
| Median Recommended Sample | 8.2 | 69.0 |
| Size Per Treatment | | |

### Example 2: Obtaining Culture of Plant Growth Promoting Pseudomonas Derived from Soybean Cultivar 5002T

### Introduction

Diazotrophs are microbes capable of residing within soybean, Glycine max, as well as other legumes to provide usable nitrogen. This macronutrient is essential for soybean's plant health, yield, and resilience (Dashti, 1998). Typically, a greater diazotrophic foundation within the root nodules supports usable nitrogen concentration and uptake (Williams, 1983). Although this varies by species and strains of Bradyrhizobium, a natural community variability in nodules occurs (Abel, 1964). Within this microbial community, Pseudomonas spp. reside in the interior of soybean nodules along with Bradyrhizobium spp, among various cultivars (Sharaf, 2019)

Although the described Pseudomonas spp. are not considered diazotrophs, their presence within the nodule interior provides the opportunity for soybean to receive plant growth promoting rhizobacteria (PGPR) benefits. It is common for Pseudomonas spp. to exhibit the ability to release organic acids capable of solubilizing phosphorus (Trivedi, 2008) as well as other nutrients. Additionally, iron uptake improves from Pseudomonas populations in soybean by bacterial siderophore production (Sharma, 2003). Furthermore, some Pseudomonas are exemplary PGPR microbes with the ability to act as biocontrol by producing anti-pathogenic compounds as well as the ability to increase growth by the production of indole acetic acid (Karnwal, 2009).

Soybean cultivars are selectively bred to present desired characteristics, for example, high yield or drought tolerance, by crossing existing breeds with these traits (Pantalone, 2017). With many cultivars suitable for a variety of environmental conditions, it is essential to derive PGPR capable of improving growth for a variety of cultivars. In this study, the Pseudomonas PGPR culture was derived from a single cultivar and sample, 5002T, but shows evidence of promoting the growth of various soybean cultivars (Griggs, 2021). Eight other cultivars tested did not allow for the extraction of and isolation of bacteria, most notably the growth promoting bacteria described below, and so 5002T grown under relatively dry growing conditions is considered a source plant for these PGPR.

### Methods

On Virginia Tech's Kentland Farm, nine diverse cultivars were studied using a split-plot design with changes in water availability. Each cultivar was replicated 3×under two different water regimes (irrigated and non-irrigated; n=2). Each cultivar by water treatment was thus replicated 3× to arrive at 54 samples. The 54 samples were allowed 5 months of growth before harvest. Following harvest, nodules were removed from the roots. This is achieved by taking forceps and grasping the nodule off of the root and placing it in a 2 mL tube for storage at −20° C. Nodules were utilized for DNA extraction to achieve a relative abundance of each bacterial species residing within the interior of the nodule. Additionally, the nodules were used to grow viable bacterial colonies on selective media to culture highly abundant Pseudomonas spp. from the interior of the nodule.

The following materials and procedure achieves sterilized surface nodules and the extraction of viable bacterial colonies from its interior:

### Materials:

Autoclaved filter paper No. 2
Forceps or measuring scooper
0.9% autoclaved NaCl solution in 50 ml tube
1.00% NaClO and 0.1% Tween (bleach detergent)
Autoclaved DI Water in 50 ml in tube
1 autoclaved 2 mL tube
Tryptic Soy Agar (TSA) and KBC plates
60 Mesh Sieve
1 mL pipet
L shaped cell spreader
Waste discard beaker

### Procedure:

Add 10-15 nodules to a 2 mL tube
Outside of hood pipet saline solution into original tube
Vortex for 30 seconds (5 secs at 8, 25 secs at 4), Level 4=˜1200 rpm; Level 8˜2500 rpm
Dump nodules onto sieve and rinse with DI water
Rinse original tube with DI water and place nodules back in with forceps or scooper
Repeat steps 2-5 two more times for a total of three saline rinses
Under the hood pipet bleach detergent into the same tube
Vortex for 30 seconds (5 secs at 8, 25 secs at 4) and let sit for 30 seconds
Pipet off half of the bleach solution and discard into waste beaker
Place10 autoclaved filter papers individually flat in the hood, similar to a lily pad. Use the same filter paper for the entire time you are using the bleach detergent.
Dump nodules onto filter paper
Aseptically place nodules back into original tube with sterile forceps or scooper
Repeat steps 7-12 once more with a new filter paper funnel for a total of two bleach rinses
Under the hood pipet DI water into the original tube
Vortex for 30 seconds (5 secs at 8, 25 secs at 4)
Dump nodules onto new filter paper funnel
Place nodules back into original tube with sterile forceps or scooper
Repeat steps 14-16 once more for a total of two DI water rinses. Use the same filter paper.
Dump all nodules onto TSA plate and roll them around the agar surface to test surface sterility
Place nodules into new tube with sterile forceps and store in −20° C. immersed in glycerol
Incubate for 5 days at 28° C.
After 5 days, check TSA plate for growth
Bacterial Extraction once the TSA, a general media, exhibits no growth use a sterile L shaped cell spreader to smash the nodules within the glycerol.
Vortex for 30 seconds on 5
Remove 100 microliters of the glycerol solution and spread it on a KBC plate using a sterile L shaped cell spreader.
Incubate for 48 hours at 28° C. and observe Pseudomonas growth

### Results and Discussion

This procedure was preformed on a harvested sample belonging to the cultivar 5002T (from a non-irrigated “drought” plot) from the described study. KBC is a selective media for Pseudomonas (Mohan & Schaad, 1987). It is important to note the use of KB+nitro agar plates allow for the growth of Bradyrhizobium, and should not be used for the selection of Pseudomonas. Sterility tests are essential in determining the surface sterility of the nodule to ensure all bacterial growth are from the interior of the nodule.

Cultured KBC plates were tested for fluorescence using a UV light table. Fluorescence is a common characteristic of growth promoting Pseudomonas among multiple strains, and of those capable of providing phosphate solubilization (Vyas & Gulati, 2009).

Using the determined abundance of the 5002T interior nodule microbiome community from DNA extraction in Sharaf's 2019 study, the 46th most abundant Pseudomonas strain was cultured on a KBC plate. Although this is not a highly abundant species, up to this point it was found difficult to culture higher abundant species. However, we knew this bacteria and others were present based on identification of DNA in the sample. This is common as the overwhelming majority of soil microbes have not been cultured in a laboratory environment (Stewart, 2012). This inability to culture bacteria from a community of microbes is still considered the state of the science today (Williams, Mark A).

The above picture illustrates the process of developing the mixed culture from the sample belonging to 5002T. Three KBC agar plates included growth of Pseudomonas spp. that were utilized in future soybean growth promoting studies. The plate on the left was a 1 mL spread of a liquid culture derived from nodules that had been surface sterilized and selected for growth. The selected colony was affectionately named “Pancake” after its morphology resembling the initial pouring of pancake batter onto a skillet. The purpose of the second streak plate, in the middle was originally intended to serve as an additional check to assess if it was a single strain. Multiple colonies were found and thus indicating the solution was composed of morphologically different types or strains of Pseudomonas. The plate on the right is from the serial dilution that was believed to be contaminated. However, after studying the plate, the suspected contaminated bacterial growth was identified as Pseudomonas that were extracted from the original sample plate. These Pseudomonads identified and shown to be morphologically and genetically (genomes) different, also include other unique isolates “Bullseye,” “Jellyfish,” and “Starfish,” which were named after their morphology on the original KBC plate.

Finally, after concluding there has been successful laboratory growth of four Pseudomonas spp., more studies were conducted to test their abilities as plant-growth promoting rhizobacteria. It is hypothesized that since these four strains were derived from a mixture of nodules, the >50 Pseudomonas variants identified, they may support soybean growth, or more likely support growth with already naturally occurring diazotrophs (Rhizobia) that must be pre-existing and present within the nodules. Pancake, when inoculated with soybean in a natural soil from Kentland Farm, Va, resulted in greater plant growth than uninoculated plants. Bullseye similarly supported greater soybean growth, but the other strains Jellyfish and Starfish did not show any evidence of growth support to soybean. This result showing no growth for these other wo bacteria Jellyfish and Starfish suggest no increased growth supported by these two bacteria. (Fredrickson, 2013).

It is possible these bacteria grow together on a culture plate because they are very similar except for a few genomic regions, and thus respond identically to culture conditions. They may also grow naturally in co-culture and may tend to grow together for that reason, but herein it is shown that Pancake and sometimes Bullseye was advantageous for supporting plant growth. Often bacteria that are the species but different variants (or strains), often have different effects on plant growth. Indeed, it has been described many times in the nodules of numerous legumes that strains of bacteria can have widely different effects (Checcucci et al. 2016; Griesmann et al., 2018).

Similarly, if correctly stabilized in the correct mixture of ingredients in solution, the bacterial mixture could also be considered a novel composition of matter.

### REFERENCES

Abel G H, Erdman L W Response of Lee Soybeans to different strains of Rhizobium Japonicum. Agron J. 1964:56:423.
Dashti N. Zhang F. Hynes R. Smith D L. Plant growth promoting rhizobacteria accelerate nodulation and increase nitrogen fixation activity by field grown soybean [Glycine max (L.) Merr.] under short season conditions. Plant Soil. 1998; 200:205-13.
Checcucci, A., Azzarello, E., Bazzicalupo, M., Galardini, M., Lagomarsino, A., Mancuso, S., Marti, L., Marzano, M. C., Mocali, S., Squartini, A. and Zanardo, M., 2016. Mixed nodule infection in Sinorhizobium meliloti-Medicago sativa symbiosis suggest the presence of cheating behavior. Frontiers in plant science, 7, p. 835.
Frederickson, M. E., 2013. Rethinking mutualism stability: cheaters and the evolution of sanctions. The Quarterly review of biology, 88(4), pp. 269-295.
Griesmann, M., Chang, Y., Liu, X., Song, Y., Haberer, G., Crook, M. B., Billault-Penneteau, B., Lauressergues, D., Keller, J., Imanishi, L. and Roswanjaya, Y. P., 2018. Phylogenomics reveals multiple losses of nitrogen-fixing root nodule symbiosis. Science, 361(6398).
Griggs R., Sharaf H., Doyle C., Williams, M. A. Pseudomonas spp. Isolated from Soybean Nodules Promote Soybean Growth and Nodulation. 2021. To be submitted to Journal Rhizosphere.
Karnwal, A. 2009. Production of indole acetic acid by fluorescent Pseudomonas in the presence of L-tryptophan and rice root exudates. Journal of Plant Pathology. 61-63.
Mohan & Schaad (1987) Mohan S K, Schaad N W. An improved agar plating assay for detecting Pseudomonas syringae pv. syringae and P. s. pv. phaseolicola in contaminated bean seed. Phytopathology. 1987; 77:1390-1395. doi: 10.1094/Phyto-77-1390.
Pantalone, V., Smallwood, C. and Fallen, B. (2017), Development of ‘Ellis’ Soybean with High Soymeal Protein, Resistance to Stem Canker, Southern Root Knot Nematode, and Frogeye Leaf Spot. Journal of Plant Registrations, 11: 250-255.
Stewart E. J. (2012). Growing unculturable bacteria. Journal of bacteriology, 194(16), 4151-4160. https://doi.org/10.1128/JB.00345-12
Sharma, S. B., Sayyed, R. Z., Trivedi, M. H., & Gobi, T. A. (2013). Phosphate solubilizing microbes: sustainable approach for managing phosphorus deficiency in agricultural soils. SpringerPlus, 2(1), 587.
Trivedi, P., and Sa, T. 2008. Pseudomonas corrugata (NRRL B-30409) mutants increased phosphate solubilization, organic acid production, and plant growth at lower temperatures. Current microbiology. 56(2): 140-144.
Vyas, P., and Gulati, A. 2009. Organic acid production in vitro and plant growth promotion in maize under controlled environment by phosphate-solubilizing fluorescent Pseudomonas. BMC microbiology. 9(1): 174.
Williams L E, Phillips D A. Increased soybean productivity with a Rhizobium japonicum Mutant1. Crop Sci. 1983; 23:246.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising a microbial strain, an extract produced by culturing the microbial strain, or any combination thereof, and at least one excipient, diluent, or carrier,
wherein the microbial strain comprises a genome having DNA sequence exhibiting at least 85% sequence identity to one or more of SEQ ID NOs: 1-31,
wherein the microbial strain comprises a genome having DNA sequence exhibiting at least 85% sequence identity to one or more of SEQ ID NOs:32-76,
wherein the microbial strain comprises a genome having DNA sequence exhibiting at least 85% sequence identity to one or more of SEQ ID NOs:77-107, or
wherein the microbial strain comprises a genome having DNA sequence exhibiting at least 85% sequence identity to one or more of SEQ ID NOs: 108-137.

2. The composition of claim 1, wherein the microbial strain is deposited with the WDCM as WDCM1247, WDCM1248, or WDCM1247.

3. The composition of claim 1, wherein the microbial strain is frozen, lyophilized, or is present as metabolically active cells.

4. The composition of claim 1, wherein the microbial strain is present in an amount of from about 109 to about 103 colony forming units per mL.

5. The composition of claim 1, wherein the microbial strain is present in an amount of from about 109 to about 108 colony forming units per mL.

6. The composition of claim 1, wherein at least one excipient, diluent, or carrier confers increased stability, wettability, dispersibility, or adherence to a substrate relative to a composition lacking the carrier.

7. The extract of claim 1, wherein the extract is produced by:
(a) culturing the microbial strain in a culture medium; and
(b) filtering the culture medium.

8. The composition of claim 1, wherein the composition comprises an emulsion, a colloid, a granule, a pellet, a powder, a spray, a suspension, or a solution.

9. The composition of claim 1, wherein at least one excipient, diluent, or carrier comprises a surfactant, a solvent, an emulsifier, a buffer, a cryoprotectant, a salt, microbial culture medium, a wetting agent, a bulking agent, an anti-caking agent, a thickener, a clay, a mineral, a lipid, a gum, a dye or colorant, a biological waste material, or any combination thereof.

10. The composition of claim 9, wherein the surfactant comprises polysorbate 20.

11. The composition of claim 9, wherein the solvent comprises water.

12. The composition of claim 1, wherein the composition further comprises an effective amount of at least one additional active ingredient.

13. The composition of claim 12, wherein the additional active ingredient comprises a fertilizer, a pesticide, an herbicide, or any combination thereof.

14. A crop legume seed coated with the composition of claim 1.

15. The crop legume seed of claim 14, wherein the seed is a soybean or edamame seed.

16. The crop legume seed of claim 14, wherein the composition further comprises a binder, filler, adhesives, adjuvant, thickener, or any combination thereof.

17. A method for increasing crop legume plant growth, the method comprising applying the composition of claim 1 to a soybean plant or seed, so that the treated plant or plant derived from the treated seed has increased growth compared to an untreated plant or plant derived from an untreated seed.

18-27. (canceled)

28. A plant treated using the method of claim 17.

29. A method for extracting plant growth promoting rhizobacteria (PGPR) from nodules of a crop legume plant, comprising
(a) mechanically cleaning the external surface of the nodules to remove soil and debris without damaging the nodule barrier that protects the nodule interior;
(b) sterilizing the surface of the nodules with a bleach and ethanol solution to lyse and kill bacterial cells and denature DNA;
(c) washing the nodules to remove the bleach and ethanol by repeated washings;
(d) testing surface of nodules to ensure sterility, wherein if the nodules are not shown to be sterile then repeat sterilization and washing steps;
(e) extracting endophytes from the nodule and plating them on KBC to grow Pseudomonas spp.; and
(f) selecting colonies for Pseudomonas with different morphologies and streaking these colonies to isolate PGPR bacterial variants.
